(19) United States
(12) Patent Application Publication
(10) Pub. No.: US 2022/0293227 A1
(43) Pub. Date: Sep. 15, 2022

(54) METHOD, APPARATUS, AND COMPUTER-READABLE MEDIUM FOR ADAPTIVE NORMALIZATION OF ANALYTE LEVELS

(71) Applicant: SomaLogic Operating Co., Inc., Boulder, CO (US)

(72) Inventors: Eduardo Daniel Tabacman, St. Paul, MN (US); Dominic Anthony Zichi, Boulder, CO (US); Matthew Joel Westacott, Thornton, CO (US); Darryl Jon Perry, Denver, CO (US)

(21) Appl. No.: 17/631,860

(22) PCT Filed: Jul. 24, 2020
(86) PCT No.: PCT/US20/43614

§ 371 Date: Jan. 31, 2022

Related U.S. Application Data
(60) Provisional application No. 62/880,791, filed on Jul. 31, 2019.

(51) Int. Cl. G16H 10/40; G01N 33/68; G16H 10/60

(52) U.S. Cl. CPC G16H 10/40; G01N 33/6848; G16H 10/60

(57) ABSTRACT

A method, apparatus, and computer-readable medium for adaptive normalization of analyte levels in one or more samples, the method including receiving one or more analyte levels corresponding to one or more analytes detected in the one or more samples, each analyte level corresponding to a detected quantity of that analyte in the one or more samples; and iteratively applying a scale factor to the one or more analyte levels over one or more iterations until a change in the scale factor between consecutive iterations is less than or equal to a predetermined change threshold or until a quantity of the one or more iterations exceeds a maximum iteration value, each iteration in the one or more iterations comprising: determining a distance between each analyte level in the one or more analyte levels and a corresponding reference distribution of that analyte in a reference data set; determining the scale factor based at least in part on analyte levels that are within a predetermined distance of their corresponding reference distributions; and normalizing the one or more analyte levels by applying the scale factor.

### CROSS-REFERENCE TO RELATED APPLICATIONS

The present application claims priority to U.S. provisional application No. 62/880,791, filed Jul. 31, 2019, the entirety of which is incorporated herein by reference.

### BACKGROUND

Median normalization was developed to remove certain assay artifacts from data sets prior to analysis. Such normalization can remove sample or assay biases that may be due to differences between samples in overall protein concentration (due to hydration state, for example), pipetting errors, changes in reagent concentrations, assay timing, and other sources of systematic variability within a single assay run. In addition, it has been observed that proteomic assays (e.g., aptamer-based proteomic assays) may produce correlated noise, and the normalization process largely mitigates these artifactual correlations.

Median normalization relies on the notion that true biological biomarkers (related to underlying physiology) are relatively rare so that most protein measurements in highly multiplexed proteomic assays are unchanged in the populations of interest. Therefore, the majority of protein measurements within a sample and across the population of interest can be considered to be sampled from a common population distribution for that analyte with a well-defined center and scale. When these assumptions don't hold, median normalization can introduce artifacts into the data, muting true biological signals and introducing systematic differences in analytes that are not differentially expressed within the sample set.

Certain pre-analytical variables related to sample collection and processing have been observed to violate the assumptions of median normalization since large numbers of analytes can be affected by under spinning samples or allowing cells to lyse prior to separation from the bulk fluid. Additionally, protein measurements from patients with chronic kidney disease have shown that many hundreds of protein levels are affected by this condition, leading to a build-up of circulating protein concentrations in these individuals compared to someone with properly functioning kidneys

Accordingly, there is a need for improvements in systems for guarding against introducing artifacts in data due to sample collection artifacts or excessive numbers of disease related proteomic changes while properly removing assay bias and decorrelating assay noise.

### BRIEF DESCRIPTION OF THE DRAWINGS

FIG. 1 illustrates a flowchart for determining the scale factor based at least in part on analyte levels that are within a predetermined distance of their corresponding reference distributions according to an exemplary embodiment.

FIG. 2 illustrates an example of a sample 200 having multiple detected analytes including 201A and 202A according to an exemplary embodiment including reference distribution 1 and reference distribution 2, respectively.

FIG. 3 illustrates the process for each iteration of the scale factor application process according to an exemplary embodiment.

FIGS. 4A-4F illustrate an example of the adaptive normalization process for a set of sample data according to an exemplary embodiment.

FIGS. 5A-5E illustrate another example of the adaptive normalization process that requires more than one iteration according to an exemplary embodiment.

FIGS. 6A-6B illustrates the analyte levels for all samples after one iteration of the adaptive normalization process described herein.

FIG. 7 illustrates the components for determining a value of the scale factor that maximizes a probability that analyte levels that are within the predetermined distance of their corresponding reference distributions are part of their corresponding reference distributions according to an exemplary embodiment.

FIGS. 8A-8C illustrate the application of Adaptive Normalization by Maximum Likelihood to the sample data in sample 4 shown in Figs.

FIGS. 9A-9F illustrate the application of Population Adaptive Normalization to the data shown in FIGS. 10A-10B according to an exemplary embodiment.

FIG. 9 illustrates another method for adaptive normalization of analyte levels in one or more samples according to an exemplary embodiment.

FIG. 10 illustrates a specialized computing environment for adaptive normalization of analyte levels according to an exemplary embodiment.

FIG. 11 illustrates median coefficient of variation across all aptamer-based proteomic assay measurements for 38 technical replicates.

FIG. 12 illustrates the Kolmogorov-Smirnov statistic against a gender specific biomarker for samples with respect to maximum allowable iterations.

FIG. 13 illustrates the number of QC samples by SampleID for plasma and serum used in analysis.

FIG. 14 illustrates the concordance of QC sample scale factors using median normalization and ANML

FIG. 15 illustrates CV Decomposition for control samples using median normalization and ANML. Lines indicate empirical cumulative distribution function of CV for each control samples within a plate (intra) between plates (inter) and total.

FIG. 16 illustrates median QC ratios using median normalization and ANML.

FIG. 17 illustrates QC ratios in tails using median normalization and ANML.

FIG. 18 illustrates scale factor concordance in time-to-spin samples using SSAN and ANML

FIG. 19 illustrates median analyte CV's across 18 donors in time-to-spin under varying normalization schemes.

FIG. 20 illustrates a concordance plot between scale factors from Covance (plasma) using SSAN and ANML.

FIG. 21 shows the distribution of all pairwise analyte correlations for Covance samples before and after ANML.

FIG. 22 illustrates a comparison of distributions obtained from data normalized through several methods.

FIG. 23 illustrates metrics for smoking logic-regression classifier model for hold-out test set using data normalized with SSAN and ANML.

FIG. 24 illustrates Empirical CDFs for c-Raf measurements in plasma and serum samples colored by collection site.

FIG. 25 illustrates concordance plots of scale factors using standard median normalization vs. adaptive median normalization in plasma (top) and serum (bottom).

FIG. 26 illustrates CDFs by site for an analyte that is not affected by the site differences for the standard normalization scheme and adaptive normalization.

FIG. 27 illustrates plasma sample median normalization scale factors by dilution and Covance collection site.

FIG. 28 where the distributions of median normalization scale factors are shown for increasing stringency in adaptive normalization

FIG. 29 shows typical behavior for a analyte which shows significant differences in RFU as a function of time-to-spin.

FIG. 30 illustrates median normalization scale factors by dilution with respect to time-to-spin.

FIG. 31 summarizes the effect of adaptive normalization on median normalization scale factors vs. time-to-spin.

FIG. 32 illustrates standard median normalization scale factors by dilution and disease state partitioned by GFR value.

FIG. 33 illustrates median normalization scale factors by dilution and disease state by standard median normalization (top) and adaptive normalization by cutoff

FIG. 34 illustrates this with the CDF of Pearson correlation of all analytes with GFR (log/log) for various normalization procedures.

FIG. 35 illustrates the distribution of inter-protein Pearson correlations for the CKD data set for unnormalized data, standard median normalization and adaptive normalization.

### DETAILED DESCRIPTION

While methods, apparatuses, and computer-readable media are described herein by way of examples and embodiments, those skilled in the art recognize that methods, apparatuses, and computer-readable media for adaptive normalization of analyte levels are not limited to the embodiments or drawings described. It should be understood that the drawings and description are not intended to be limited to the particular forms disclosed. Rather, the intention is to cover all modifications, equivalents and alternatives falling within the spirit and scope of the appended claims. Any headings used herein are for organizational purposes only and are not meant to limit the scope of the description or the claims. As used herein, the word “can” is used in a permissive sense (i.e., meaning having the potential to) rather than the mandatory sense (i.e., meaning must). Similarly, the words “include,” “including,” “includes”, “comprise,” “comprises,” and “comprising” mean including, but not limited to.

Applicant has developed a novel method, apparatus, and computer-readable medium for adaptive normalization of analyte levels detected in samples. The techniques disclosed herein and recited in the claims guard against introducing artifacts in data due to sample collection artifacts or excessive numbers of disease related proteomic changes while properly removing assay bias and decorrelating assay noise.

This disclosed adaptive normalization techniques and systems remove affected analytes from the normalization procedure when collection biases exist within the populations of interest or an excessive number of analytes are biologically affected in the populations being studied, thereby preventing the introduction of bias into the data.

The directed aspect of adaptive normalization utilizes definitions of comparisons within the sample set that may be suspect for bias. These include distinct sites in multisite sample collections that have been shown to exhibit large variations in certain protein distributions and key clinical variates within a study. A clinical variate that can be tested is the clinical variate of interest in the analysis, but other confounding factors may exist.

The adaptive aspect of adaptive normalization refers to the removal of those analytes from the normalization procedure that are seen to be significantly different in the directed comparisons defined at the outset of the procedure. Since each collection of clinical samples is somewhat unique, the method adapts to learn those analytes necessary for removal from normalization and sets of removed analytes will be different for different studies.

Additionally, by removing affected analytes from median normalization, the present system and method minimizes the introduction of normalization artifacts without correcting the affected analytes. To the contrary, sample handling artifacts are amplified by such analysis, as will the underlying biology in the study. These effects are discussed in greater detail in the EXAMPLES section.

The disclosed techniques for adaptive normalization follow a recursive methodology to check for significant differences between user directed groups on an analyte-by-analyte level. A dataset is hybridization normalized and calibrated first to remove initially detected assay noise and bias. This dataset is then passed into the adaptive normalization process (described in greater detail below) with the following parameters:

(1) the directed groups of interest,

(2) the test statistic to be used for determining differences among the directed groups,

(3) a multiple test correction method, and

(4) a test significance level cutoff.

The set of user-directed groups can be defined by the samples themselves, by collection sites, sample quality metrics, etc., or by clinical covariates such as Glomerular Filtration Rate (GFR), case/control, event/no event, etc. Many test statistics can be used to detect artifacts in the collection, including Student's t-test, ANOVA, Kruskal-Wallis, or continuous correlation. Multiple test corrections include Bonferroni, Holm and Benjamini-Hochberg (BH), to name a few.

The adaptive normalization process is initiated with data that is already hybridization normalized and calibrated. Univariate test statistics are computed for each analyte level between the directed groups. The data is then median normalized to a reference (Covance dataset), removing those analytes levels with significant variation among the defined groups from the set of measurements used to produce normalization scale factors. Through this adaptive step, the present system will remove analyte levels that have the potential to introduce systematic bias between the defined groups. The resulting adaptive normalization data is then used to recompute the test statistics, followed by a new adaptive set of measurements used to normalize the data, and so on.

The process can be repeated over multiple iterations until one or more conditions are met. These conditions can include convergence, i.e., when analyte levels selected from consecutive iterations are identical, a degree of change of analyte levels between consecutive iterations being below a certain threshold, a degree of change of scale factors between consecutive iterations being below a certain threshold, or a certain number of iterations passing. The output of the adaptive normalization process can be a normalized file annotated with a list of excluded analytes/analyte levels, the value of the test statistic, and the corresponding statistical values (i.e., the adjusted p-value).

As will be explained further in the EXAMPLES sections, for a dataset that includes an extreme number of artifacts—either biological or collection related—the present system is able to filter artifacts and noise that is not detected by previous median normalization schemes.

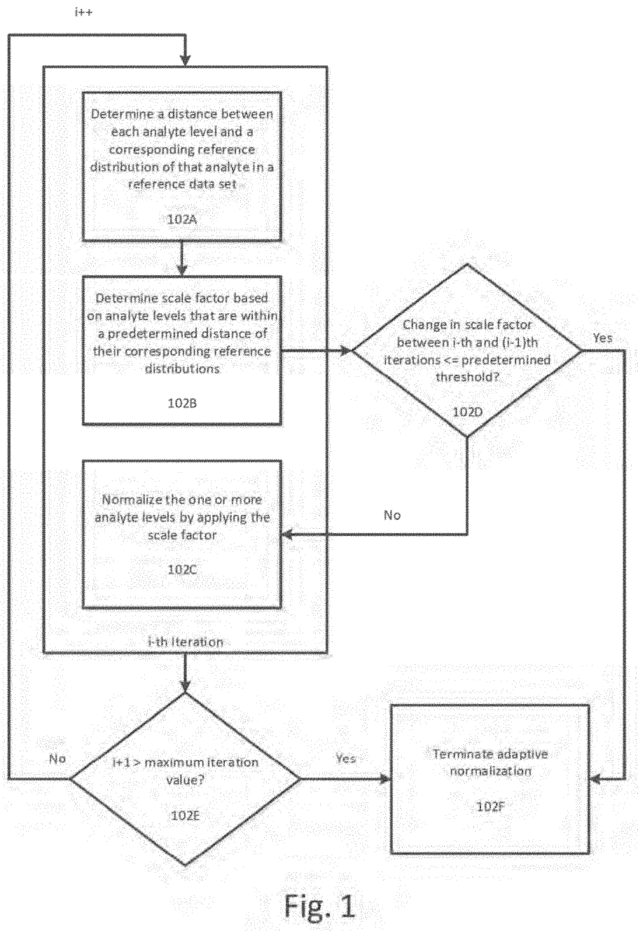

FIG. 1 illustrates a method for adaptive normalization of analyte levels in one or more samples according to an exemplary embodiment. One or more analyte levels corresponding to one or more analytes detected in the one or more samples are received. Each analyte level corresponds to a detected quantity of that analyte in the one or more samples.

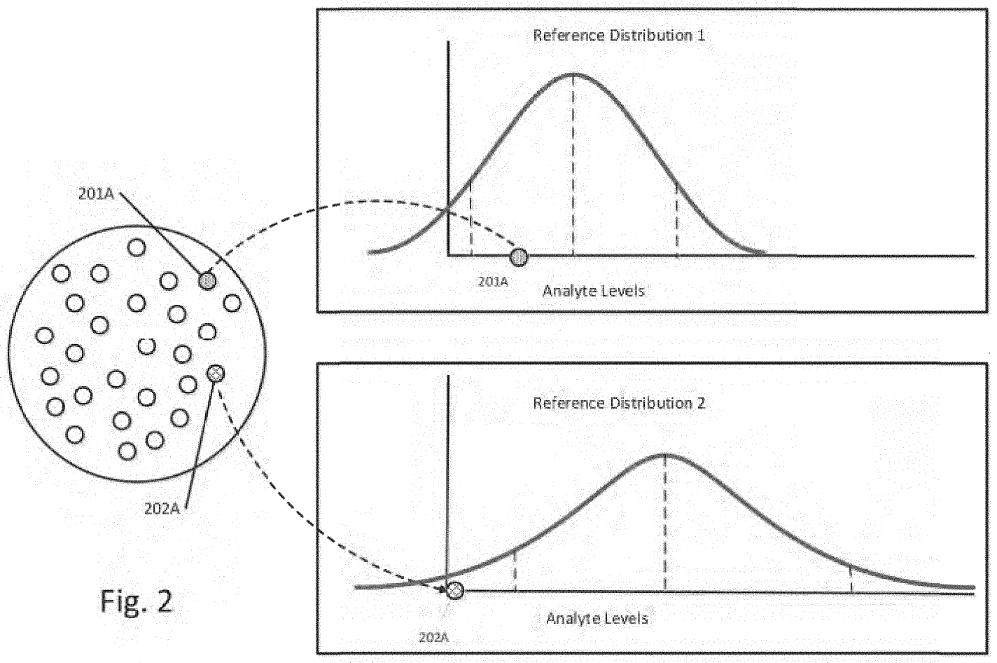

FIG. 2 illustrates an example of a sample 200 having multiple detected analytes according to an exemplary embodiment. As shown in FIG. 2, the larger circle 200 represents the sample, and each of the smaller circles represents an analyte level for a different analyte detected in the sample. For example, circles 201A and 202A correspond to two different analyte levels for two different analytes. Of course, the quantity of analytes shown in FIG. 2 is for illustration purposes only, and the number of analyte levels and analytes detected in a particular sample can vary.

As shown in FIG. 2, sample 200 includes various analytes, such as analyte 201A and analyte 202A. Reference distribution 1 is a reference distribution corresponding to analyte 201A and reference distribution 2 is a reference distribution corresponding to analyte 202A. The reference distributions can take any suitable format. For example, as shown in FIG. 2, each reference distribution can plot analyte levels of an analyte detected in a reference population or reference samples. Of course, the reference distribution can be plotted and/or stored in a variety of different ways. For example, the reference distribution can be plotted on the basis of a count of each of analyte level or range of analyte levels. Additionally, the reference distributions can be processed to extract mean, median, and standard deviation values and those stored values can be used in the distance determination process, as discussed below. Many variations are possible and these examples are not intended to be limiting.

As shown in FIG. 2, the analyte level of each analyte in the sample (such as analytes 201A and 202A) are compared to the corresponding reference distributions (such as distributions 1 and 2) either directly or via statistical measure extracted from the reference distributions (such as mean, median, and/or standard deviation) to determine the statistical and/or mathematical distance between each analyte level in the sample and the corresponding reference distribution.

The one or more samples in which the analyte levels are detected can include a biological sample, such as a blood sample, a plasma sample, a serum sample, a cerebral spinal fluid sample, a cell lysates sample, and/or a urine sample. Additionally, the one or more analytes can include, for example, protein analyte(s), peptide analyte(s), sugar analyte(s), and/or lipid analyte(s).

The analyte level of each analyte can be determined in a variety of ways. For example, each analyte level can be determined based on applying a binding partner of the analyte to the one or more samples, the binding of the binding partner to the analyte resulting in a measurable signal. The measurable signal can then be measured to yield the analyte level. In this case, the binding partner can be an antibody or an aptamer. Each analyte level can additionally or alternatively be determined based on mass spectrometry of the one or more samples.

Returning to FIG. 1, at step 102C a scale factor is iteratively applied to the one or more analyte levels over one or more iterations until a change in the scale factor between consecutive iterations is less than or equal to a predetermined change threshold 102D or until a quantity of the one or more iterations exceeds a maximum iteration value (102F).

The scale factor is a dynamic variable that is re-calculated for each iteration. By determining and measuring the change in the scale factor between subsequent iterations, the present system is able to detect when further iterations would not improve results and thereby terminate the process.

Additionally, a maximum iteration value can be utilized as a failsafe, to ensure that the scale factor application process does not repeat indefinitely (in an infinite loop). The maximum iteration value can be, for example, 10 iterations, 20 iterations, 30 iterations, 40 iterations, 50 iterations, 100 iterations, or 200 iterations.

Optionally, the maximum iteration value can be omitted and the scale factor can be iteratively applied to the one or more analyte levels over one or more iterations until a change in the scale factor between consecutive iterations is less than or equal to a predetermined change threshold, without consideration of the number of iterations required.

The predetermined change threshold can be set by a user or set to some default value. For example, the predetermined change threshold can be set to a very low decimal value (e.g., 0.001) such that the scale factor is required to reach a “convergence” where there is very little measurable change in the scale factor between iterations in order for the process to terminate.

The change in the scale factor between subsequent iterations can measured as a percentage change. In this case, the predetermined change threshold can be, for example, a value between 0 and 40 percent, inclusive, a value between 0 and 20 percent, inclusive, a value between 0 and 10 percent, inclusive, a value between 0 and 5 percent, inclusive, a value between 0 and 2 percent, inclusive, a value between 0 and 1 percent, inclusive, and/or 0 percent.

At step 102A a distance is determined between each analyte level in the one or more analyte levels and a corresponding reference distribution of that analyte in a reference data set.

This distance is a statistical or mathematical distance and can be measure the degree to which a particular analyte level differs from a corresponding reference distribution of that same analyte. Reference distributions of various analyte levels can be pre-compiled and stored in a database and accessed as required during the distance determination process. The reference distributions can be based upon reference samples or populations and be verified to be free of contamination or artifacts through a manual review process or other suitable technique.

The determination of a distance between each analyte level in the one or more analyte levels and a corresponding reference distribution of that analyte in a reference data set can include determining an absolute value of a Mahalanobis distance between each analyte level and the corresponding reference distribution of that analyte in the reference data set.

The Mahalanobis distance is a measure of the distance between a point P and a distribution D. An origin point for computing this measure can be at the centroid (the center of mass) of a distribution. The origin point for computation of the Mahalanobis distance (“M-Distance”) can also be a mean or median of the distribution and utilize the standard deviation of the distribution, as will be discussed further below.

Of course, there are other ways of measuring statistical or mathematical distance between an analyte level in the sample and a corresponding reference distribution that can be utilized. For example, determining a distance between each analyte level in the one or more analyte levels and a corresponding reference distribution of that analyte in a reference data set can include determining a quantity of standard deviations between each analyte level and a mean or a median of the corresponding reference distribution of that analyte in the reference data set.

Returning to FIG. 1, at step 102B a scale factor is determined based at least in part on analyte levels that are within a predetermined distance of their corresponding reference distributions.

This step includes a first sub-step of identifying all analyte levels in the sample that are within a predetermined distance threshold of their corresponding reference distributions. The predetermined distance that is used as a cutoff to identify analyte levels to be used in the scale factor determination process can be set by a user, set to some default value, and/or customized to the type of sample and analytes involved.

Additionally, the predetermined distance threshold will depend on how the statistical distance between the analyte level and the corresponding reference distribution is determined. In the case when an M-Distance is used, the predetermined distance can be value in a range between 0.5 to 6, inclusive, a value in a range between 1 to 4, inclusive, a value in a range between 1.5 to 3.5, inclusive, a value in a range between 1.5 to 2.5, inclusive, and/or a value in a range between 2.0 to 2.5, inclusive. The specific predetermined distance used to filter analyte levels from use in the scale factor determination process can depend on the underlying data set and the relevant biological parameters. Certain types of samples may have a greater inherent variation than others, warranting a higher predetermined distance threshold, while others may warrant a lower predetermined distance threshold.

Returning to FIG. 1. At step 102A distance is calculated between each analyte level and the corresponding reference distribution for that analyte. The corresponding reference distribution can be looked up based upon an identifier associate with the analyte and stored in memory or based upon an analyte identification process that detects each type of analyte. The distance can be calculated, for example, as an M-Distance, as discussed previously. The M-Distance be computed on the basis of the mean, median, and/or standard deviation of the corresponding reference distribution so that the entire reference distribution does not need to be stored in memory. For example, the M-Distance between each analyte level in the sample and the corresponding reference distribution can be given by:

$$M = \frac{( {x}_{p} - {μ}_{ref , p} )}{{σ}_{r e f , p}}$$

Where M is the Mahalanobis Distance (“M-Distance”), xₚ is the value of an analyte level in the sample, μ_{ref,p} is the mean of the reference distribution corresponding to that analyte, and σ_{ref,p} is the standard deviation of the reference distribution corresponding to that analyte.

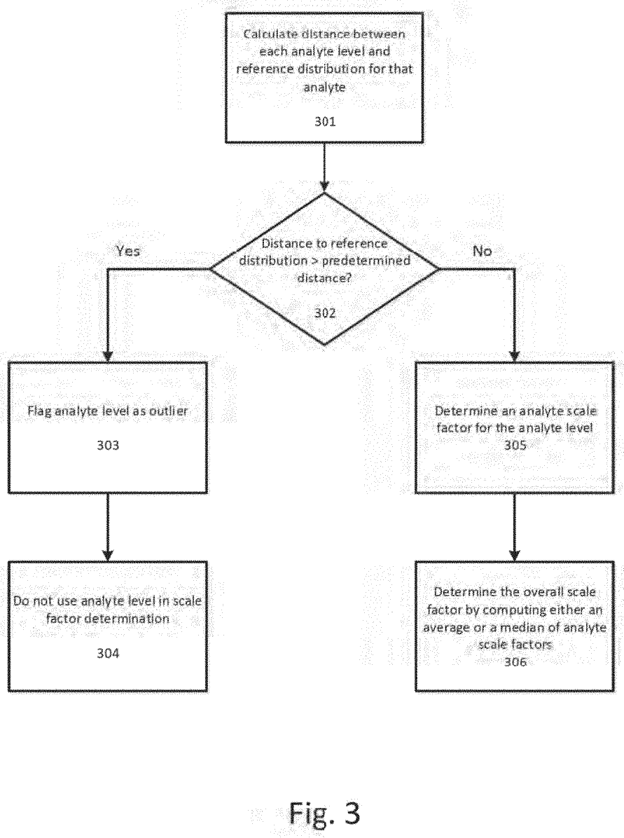

FIG. 3 illustrates a flowchart for determining the scale factor based at least in part on analyte levels that are within a predetermined distance of their corresponding reference distributions according to an exemplary embodiment.

At step 301 an analyte scale factor is determined for each analyte level that is within the predetermined distance of the corresponding reference distribution. This analyte scale factor is determined based at least in part on the analyte level and a mean or median value of the corresponding reference distribution. For example, the analyte scale factor for each analyte can be based upon the mean of the corresponding reference distribution:

$$S {F}_{Analyte} = \frac{{μ}_{ref , p}}{{x}_{p}}$$

Where SF_{Analyte} is the scale factor for each analyte that is within a predetermined distance of its corresponding reference distribution, μ_{ref,p} is the mean of the reference distribution corresponding to that analyte, and xₚ is the value of an analyte level in the sample.

The analyte scale factor can also be based upon the median of the corresponding reference distribution:

$$S {F}_{Analyte} = \frac{{\tilde{x}}_{ref , p}}{{x}_{p}}$$

Where SF_{Analyte} is the scale factor for each analyte that is within a predetermined distance of its corresponding reference distribution, \tilde{x} is the median of the reference distribution corresponding to that analyte, and xₚ is the value of an analyte level in the sample.

At step 302 the overall scale factor for the sample is determined by computing either a mean or a median of analyte scale factors corresponding to analyte levels that are within the predetermined distance of their corresponding reference distributions. The overall scale factor is therefore given by one of:

SF_{Overall}=\tilde{x}_{SF}_{_{Analyte}}

Or:

SF_{Overall}=σ_{SF}_{_{Analyte}}

Where SF_{Overall} is the overall scale factor (referred to herein as the “scale factor”) to be applied to the analyte levels in the sample, \tilde{x}_{SF}_{_{Analyte}} is the mean of the analyte scale factors, and a σ_{SF}_{_{Analyte}} is the median of the analyte scale factors.

At step 302 a determination is made whether the distance between the analyte level and the reference distribution is greater than the predetermined distance threshold. If so, the analyte level is flagged as an outlier at step 303 and the analyte level is excluded from the scale factor determination process at step 304. Otherwise, if the distance between the analyte level and the reference distribution is less than or equal to the predetermined distance threshold, then the analyte level is flagged as being within an acceptable distance at step 305 and the analyte level is used in the scale factor determination process at step 306.

The flagging of each analyte level can encoded and tracked by a data structure for each iteration of the scale factor application process, such as a bit vector or other Boolean value storing a 1 or 0 for each analyte level, the 1 or 0 indicating whether the analyte level should be used in the scale factor determination process. The corresponding data structure can the n be refreshed/re-encoded during a new iteration of the scale factor application process.

When the scale factor determination process occurs at step 306, the data structure encoding the results of the distance threshold evaluation process in steps 301-302 can be utilized to filter the analyte levels in the sample to extract and/or identify only those analyte levels to be used in the scale factor determination process.

While the origin point for computing the predetermined distance for each reference distribution is shown as the centroid of the distribution for clarity, it is understood that other origin points can be utilized, such as the mean or median of the distribution, or the mean or median adjusted based upon the standard deviation of the distribution.

Returning to FIG. 1, at step 102D a determination is made regarding whether the change in scale factor between the determined scale factor and the previously determined scale factor (for a previous iteration) is less than or equal to a predetermined threshold. If the first iteration of the scaling process is being performed than this step can be skipped. This step compares the current scale factor with the previous scale factor from the previous iteration and determines whether the change between the previous scale factor and the current scale factor exceeds the predetermined threshold.

As discussed earlier, this predetermined threshold can be some user-defined threshold, such as a 1% change, and/or can require nearly identical scale factors (˜0% change) such that the scale factor converges to a particular value.

If the change in scale factor between the i^{th} and the (i−1)^{th} iterations is less than or equal to the predetermined threshold, then at step 102F the adaptive normalization process terminates.

Otherwise, if the change in scale factor between the i^{th} and the (i−1)^{th} iterations is greater than the predetermined threshold, then the process proceeds to step 102C, where the one or more analyte levels in the sample are normalized by applying the scale factor. Note that all analyte levels in the sample are normalized using this scale factor, and not only the analyte levels that were used to compute the scale factor. Therefore, the adaptive normalization process does not “correct” collection site bias, or differential protein levels due to disease; rather, it ensures that such large differential effects are not removed during normalization since that would introduce artifacts in the data and destroy the desired protein signatures.

After the normalization step at 102C, at optional step 102E, a determination is made regarding whether repeating one more iteration of the scaling process would exceed the maximum iteration value (i.e., whether i+1>maximum iteration value). If so, the process terminates at step 102F. Otherwise, the next iteration is initialized (i++) and the process proceeds back to step 102A for another round of distance determination, scale factor determination at step 102B, and normalization at step 102C (if the change in scale factor exceeds the predetermined threshold at 102D).

Steps 102A-102D are repeated for each iteration until the process terminates at step 102F (based upon either the change in scale factor falling within the predetermined threshold or the maximum iteration value being exceeded.

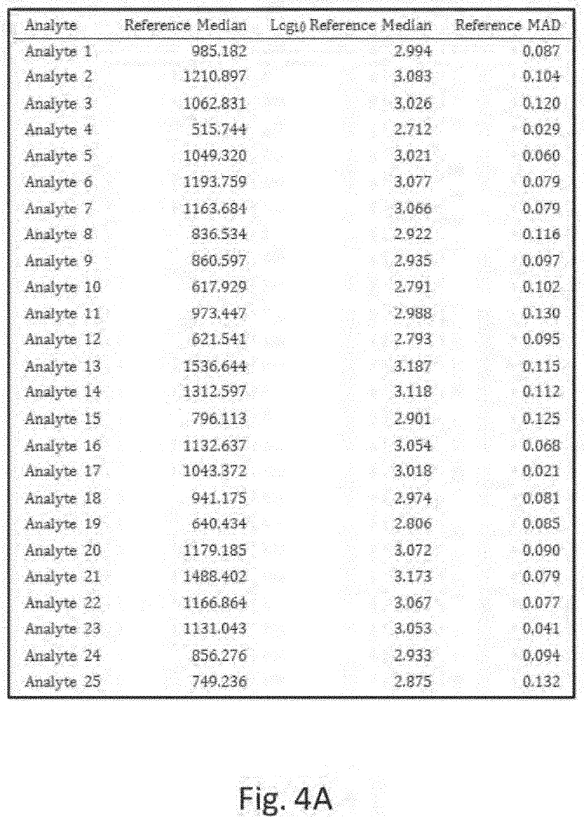

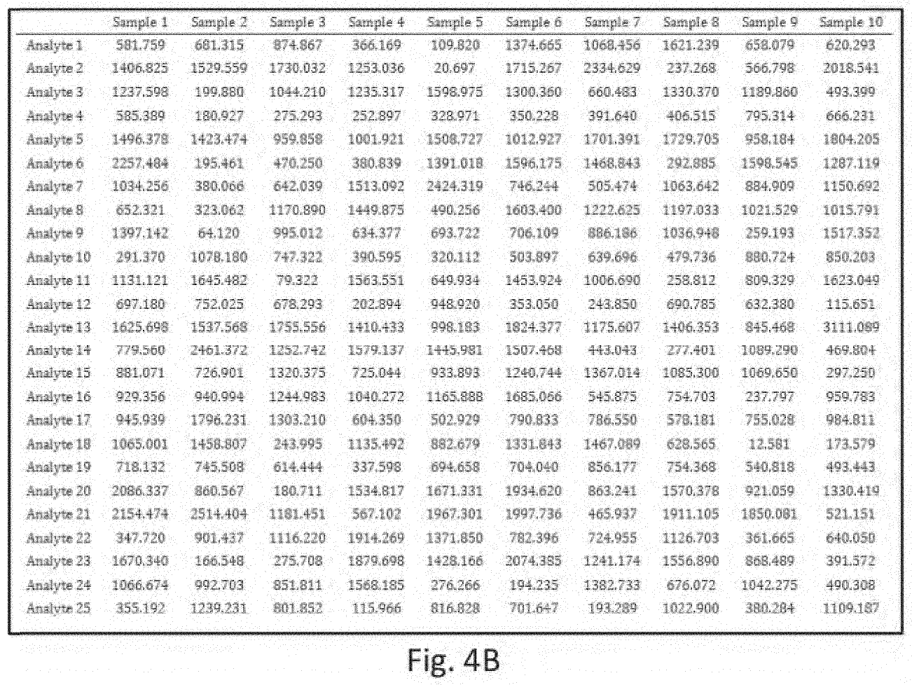

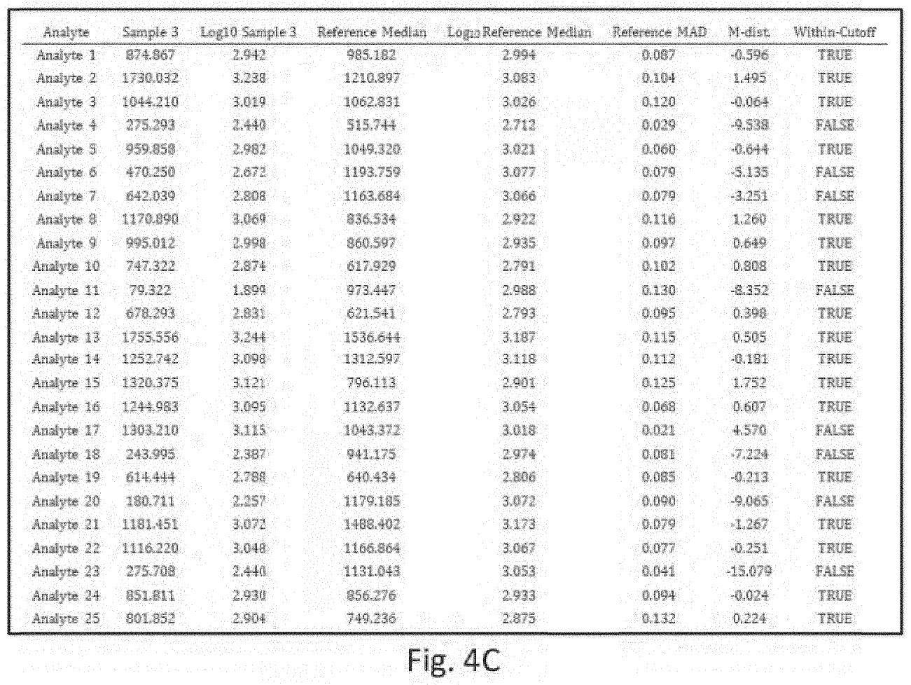

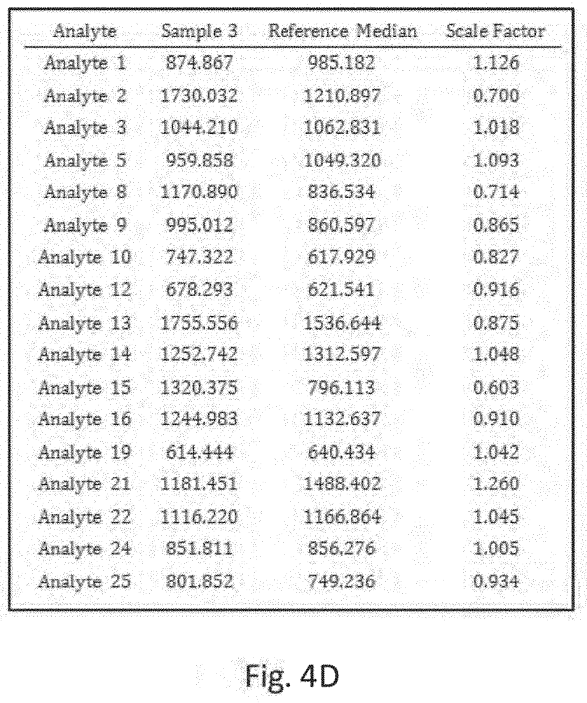

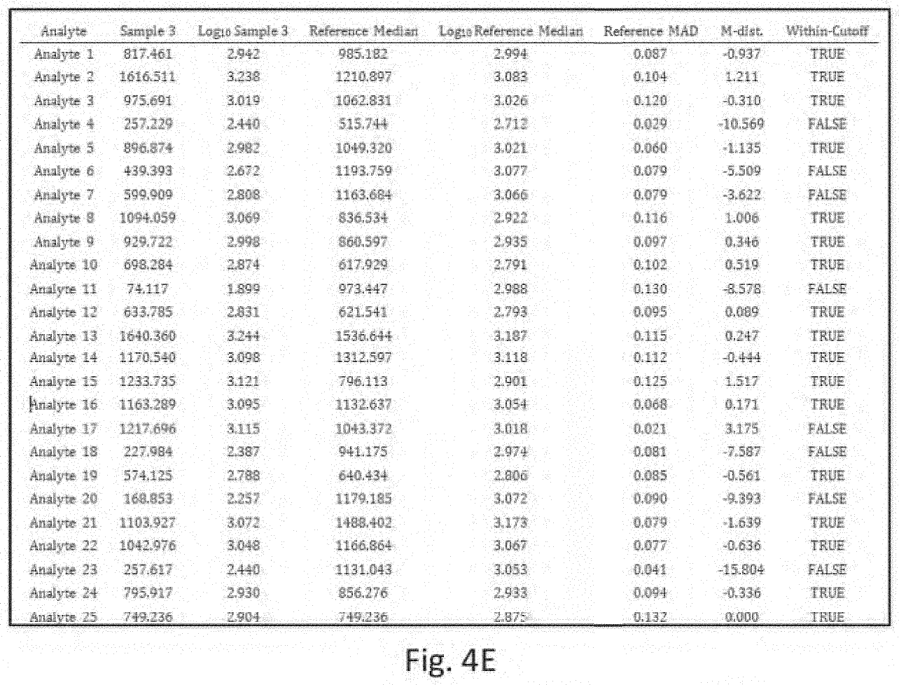

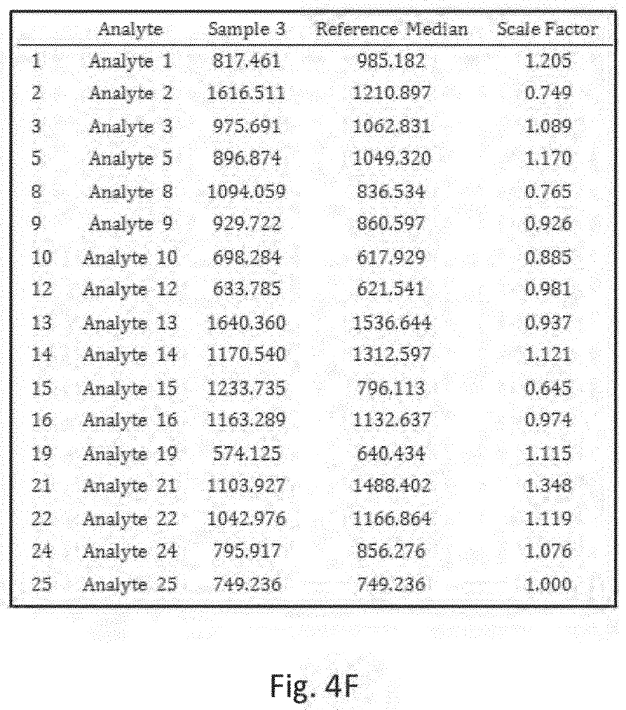

FIGS. 4A-4F illustrate an example of the adaptive normalization process for a set of sample data according to an exemplary embodiment.

FIG. 4A illustrates a set of reference data summary statistics that are to be used for both calculation of scale factors and distance determination of analyte levels to reference distributions. The reference data summary statistics summarize the pertinent statistical measures for reference distributions corresponding to 25 different analytes.

FIG. 4B illustrates a set of sample data corresponding to analyte levels of the 25 different analytes measured across ten samples. Each of the analyte levels are expressed as relative fluorescent units but is understood that other units of measurement can be utilized.

The adaptive normalization process can iterate through each sample by first calculating the Mahalanobis distance (M-Distance) between each analyte level and the corresponding reference distribution, determining whether each M-Distance falls within a predetermined distance, calculating a scale factor (both at the analyte level and overall), normalizing the analyte levels, and then repeating the process until the change in the scale factor falls under a predefined threshold.

As an example, the tables in FIGS. 4C-4F will utilize the measurements in Sample 3 in FIG. 4B. As shown in FIG. 4C, an M-Distance is calculated between each analyte level in sample 3 and the corresponding reference distribution. This M-Distance is given by the equation (discussed earlier):

$$M = \frac{( {x}_{p} - {μ}_{ref , p} )}{{σ}_{ref , p}}$$

Also shown in the table of FIG. 4C is a Boolean variable Within-Cutoff, that indicates whether the absolute value of the M-Distance for each analyte is within the predetermined distance required to be used in the scale factor determination process. In this case, the predetermined distance is set to 2. As shown in FIG. 4C, analytes 3, 6, 7, 11, 17, 18, 20, and 23 are greater than the cutoff distance of |2| and so these will not be used in the following scale factor determination step.

To determine the overall scale factor, a scale factor for each of the remaining analytes (the analytes having a Within-Cutoff value of TRUE) is determined as discussed previously. FIG. 4D illustrates the analyte scale factor for each of the analytes. The median of these analyte scale factors is then set to be the overall scale factor. Of course, the mean of these analyte scale factors can also be used as the overall scale factor.

In this case, the scale factor is given by:

SF_{Overall}=median(SF_{Analyte 1 . . . p})=0.9343

Where SF_{Analyte 1 . . . p} is the analyte scale factor for each of the analytes that are used in the scale factor determination process.

The 25 analyte measurements for sample 3 are then multiplied by this scale factor and the process is repeated. New M-Distances are calculated for this normalized data and analytes that are within the predetermined distance threshold are determined, as shown in FIG. 4E. FIG. 4F additionally illustrates the analyte scale factors for this next iteration. Using the above mentioned formula for the overall scale factor, the overall scale factor for this iteration is determined to be equal to 1 (the median of the analyte scale factors).

Since the overall scale factor is determined to be 1, the process can be terminated, since application of this scale factor will not produce any change to the data and the next scale factor will also be 1.

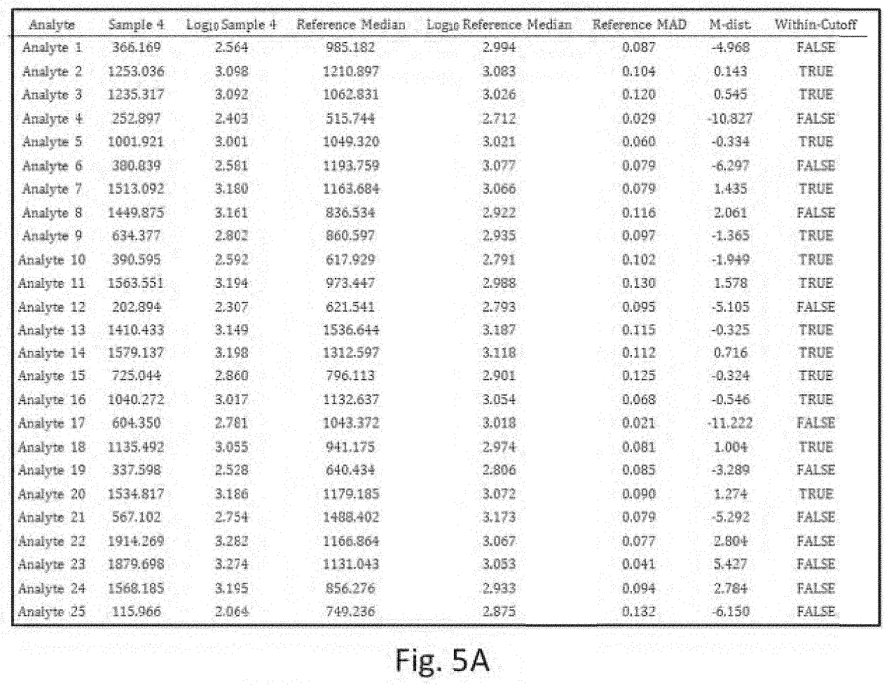

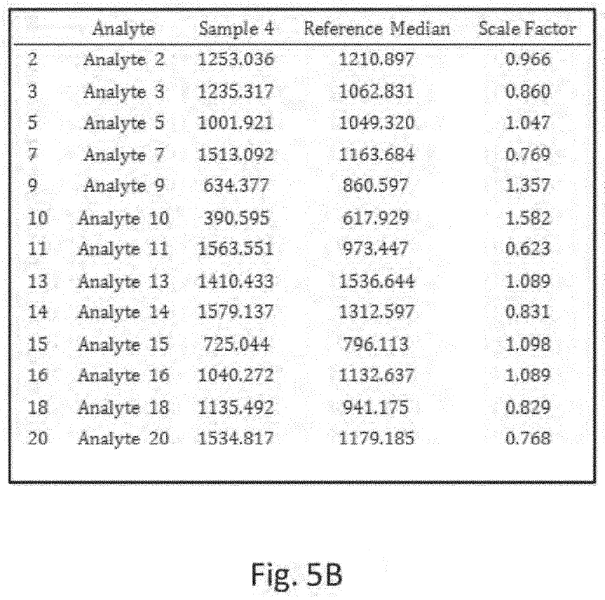

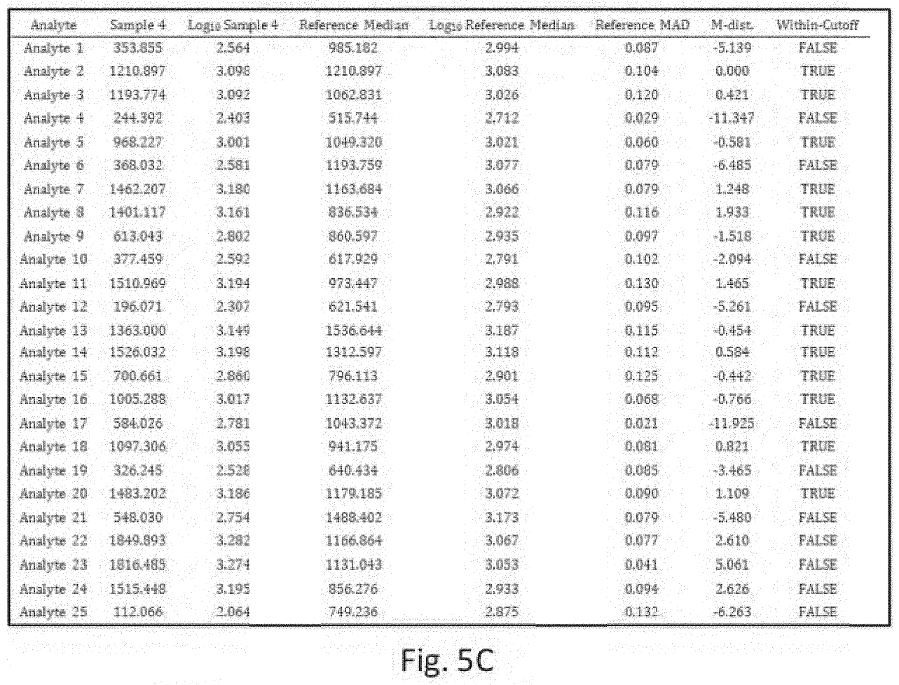

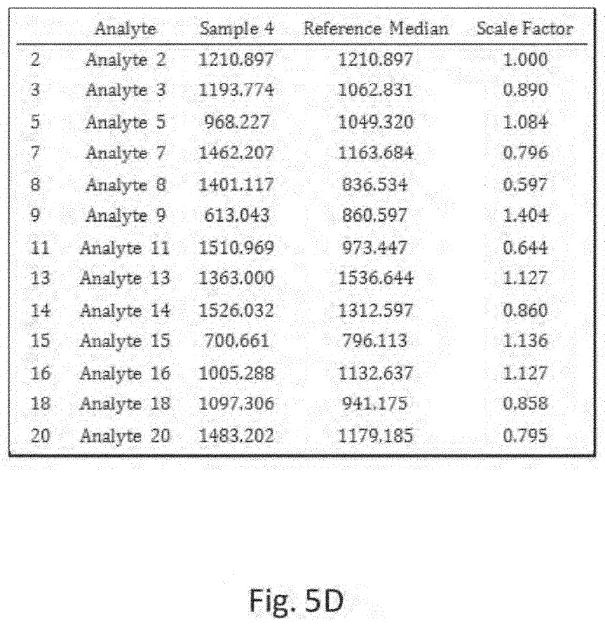

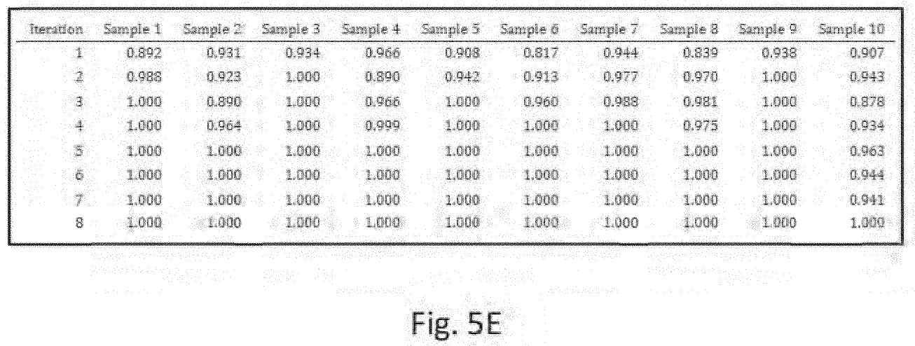

FIGS. 5A-5E illustrate another example of the adaptive normalization process that requires more than one iteration according to an exemplary embodiment. These figures use the data corresponding to sample 4 in FIGS. 4A-4B.

FIG. 5A illustrates the M-Distance values and the corresponding Boolean “Within-Cutoff” values of each of the analytes in sample 4. As shown in FIG. 5A, analytes 1, 4, 6, 8, 12, 17, 19, and 21-25 are excluded from the scale factor determination process.

FIG. 5B illustrates the analyte scale factors for each of the remaining analytes. The overall scale factor for this iteration is taken as the median of these values, as discussed previously, and is equal to 0.9663.

This scale factor is applied to the analyte levels to generate the analyte levels shown in FIG. 5C. FIG. 5C also illustrates the M-Distance determination and cutoff determination results for the second iteration of the normalization process. In this case, analytes 1, 4, 6, 10, 12, 17, 19, and 21-25 are excluded from the scale factor determination process.

FIG. 5D illustrates the analyte scale factors for each of the remaining analytes. The overall scale factor for this iteration is taken as the median of these values, as discussed previously, and is equal to 0.8903. As this scale factor has not yet converged to a value of 1 (indicating no further change in scale factor), the process is repeated until a convergence is reached (or until the change in scale factor falls within some other predefined threshold).

FIG. 5E illustrates the scale factor determined for each sample shown in FIGS. 4A-4B across eight iterations of the scale factor determination and adaptive normalization process. As shown in FIG. 5E, the scale factor for sample 4 does not converge until the fifth iteration of the process.

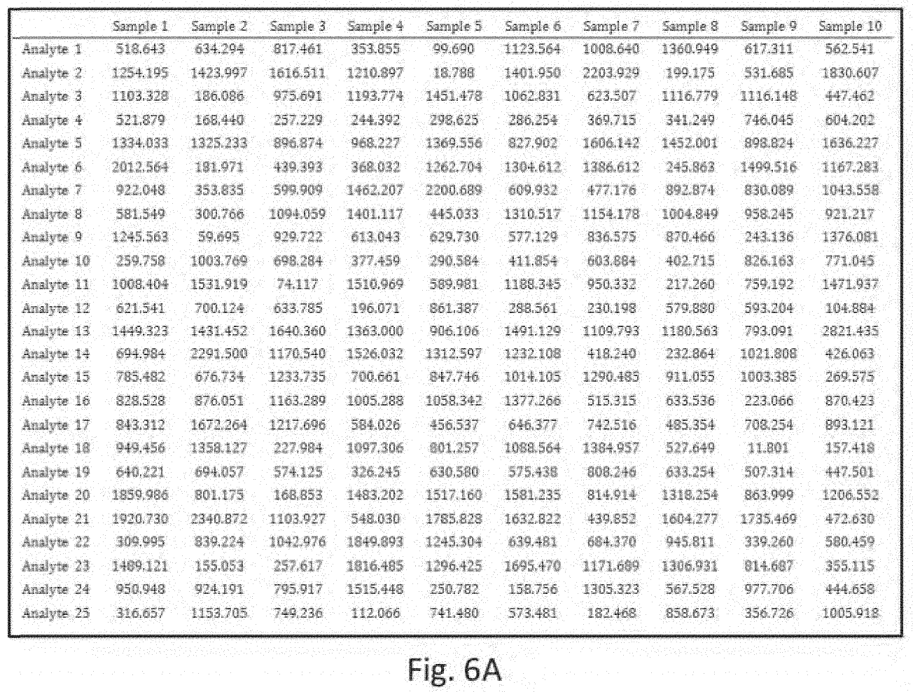

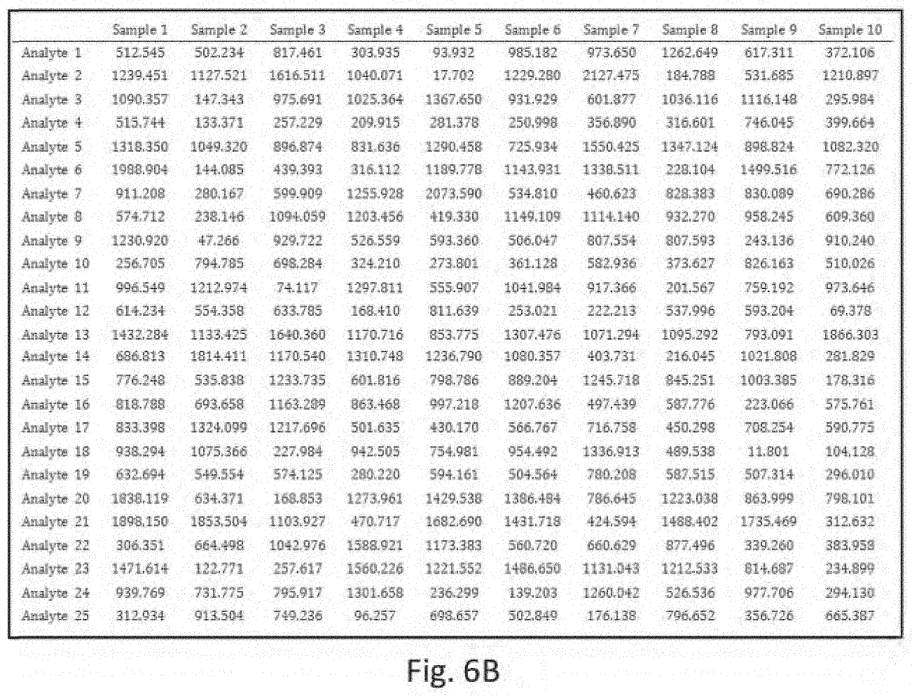

The analyte level data for each of the samples will change after each iteration (assuming the determined scale factor is not 1). For example, FIG. 6A illustrates the analyte levels for all samples after one iteration of the adaptive normalization process described herein. FIGS. 6A-6B illustrates the analyte levels for all samples after the adaptive normalization process is completed (in this example, after all scale factors have converged to 1).

Referring back to FIG. 1, the scale factor determination step 102B can be performed in other ways. In particular, determining the scale factor based at least in part on analyte levels that are within a predetermined distance of their corresponding reference distributions can include determining a value of the scale factor that maximizes a probability that analyte levels that are within the predetermined distance of their corresponding reference distributions are part of their corresponding reference distributions.

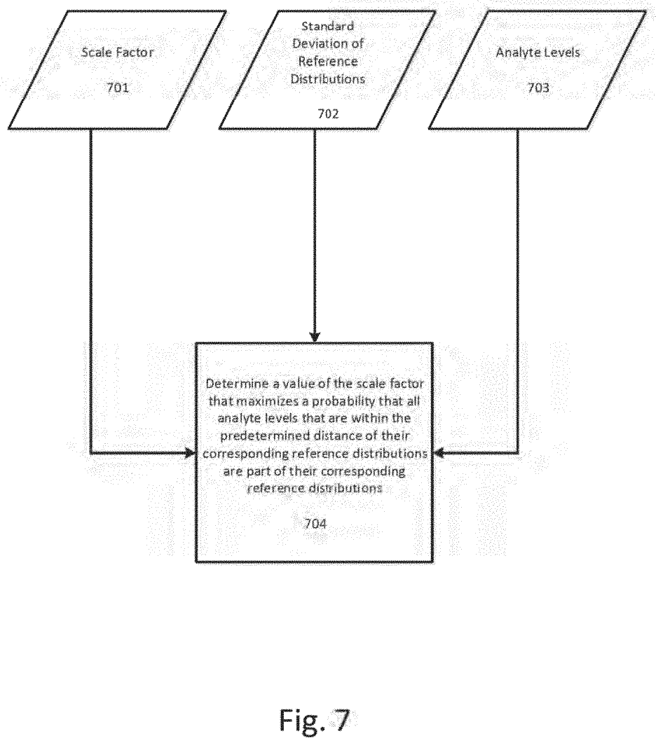

FIG. 7 illustrates the requirements for determining a value of the scale factor that maximizes a probability that analyte measurements within a given sample are derived from a reference distribution.

In this case, the probability that each analyte level is part of the corresponding reference distribution can be determined based at least in part on the scale factor, the analyte level, a standard deviation of the corresponding reference distribution, and a median of the corresponding reference distribution.

At step 704 _{[MW1]} a value of the scale factor is determined that maximizes a probability that all analyte levels that are within the predetermined distance of their corresponding reference distributions are part of their corresponding reference distributions. As shown in FIG. 7, this probability function utilizes a standard deviation of the corresponding reference distributions 702 and the analyte levels 703 in order to determine the value of the scale factor 7015 that maximizes this probability.

Adaptive normalization that uses this technique for scale factor determination is referred to herein as Adaptive Normalization by Maximum Likelihood (ANML). The primary difference between ANML and the previous technique for adaptive normalization described above (which operates on single samples and is referred to herein as Single Sample Adaptive Normalization (SSAN)), is the scale factor determination step.

Whereas medians were used to calculate the scale factor for SSAN, ANML utilizes the information of the reference distribution to maximize the probability the sample was derived from the reference distribution:

$${log}_{10} {SF}_{Overall} = \frac{{\sum }_{p = 1}^{N} ( {μ}_{ref , p} - {x}_{ref , p} ) {σ}_{ref , p}^{- 2}}{{\sum }_{p = 1}^{N} {σ}_{r e f , p}^{- 2}}$$

This formula relies on the assumption that the reference distribution follows a log normal probability. Such an assumption allows for the simple closed form for the scale factors but is not necessary. As shown above, the overall scale factor for ANML is a weighted variance average. The contribution to the scale factor, SF_{Overall}, of analyte measurements which show large population variance will be weighted less than those coming from smaller population variances.

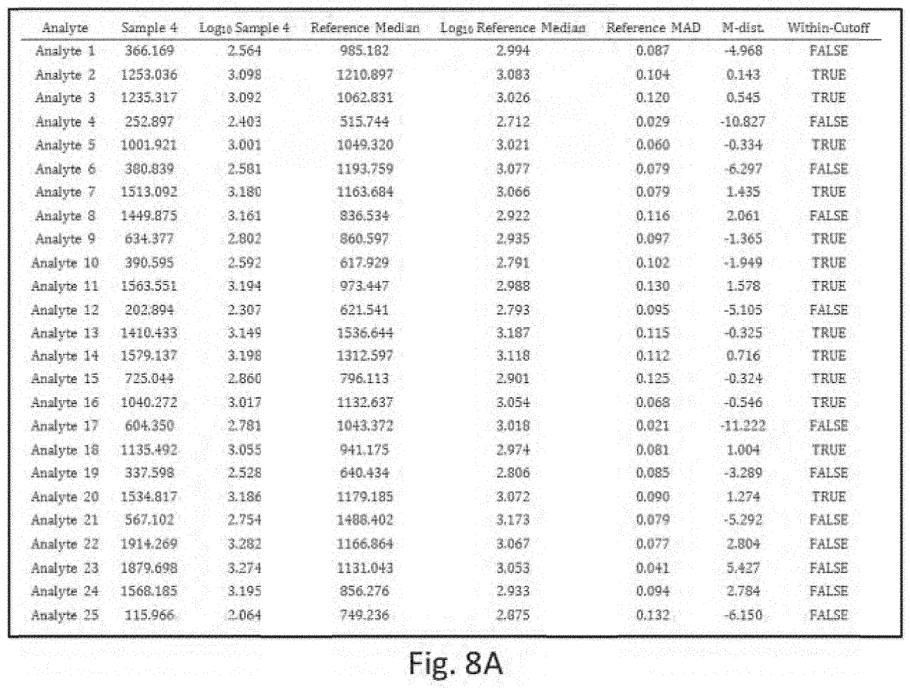

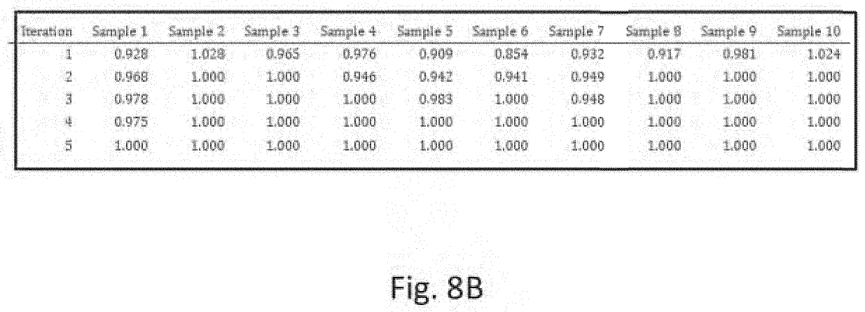

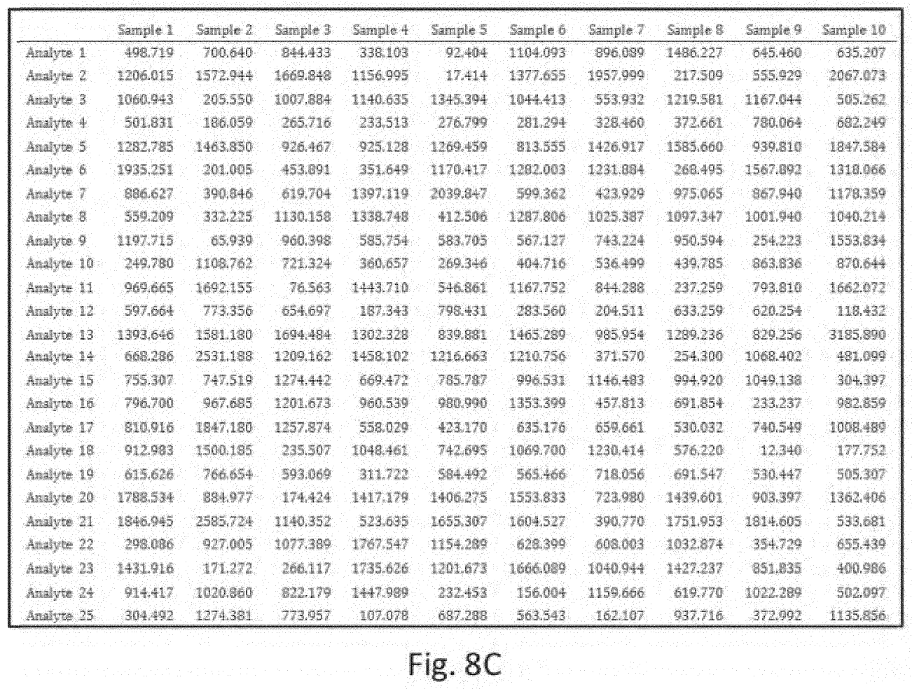

FIGS. 8A-8C illustrate the application of Adaptive Normalization by Maximum Likelihood to the sample data in sample 4 shown in FIGS. 4A-4B according to an exemplary embodiment. FIG. 4A illustrates the M-Distance values and With-Cutoff values of each analyte in a first iteration. As shown in FIG. 8A, the non-usable analytes from the first iteration for sample 4 are analytes 1, 4, 6, 8, 12, 17, 19, 21, 22, 23, 24, and 25. For the calculation of the scale factor we take the log 10 transformed reference data, standard deviation, and sample data and apply the above-mentioned equation for scale factor determination:

$${log}_{10} {SF}_{Overall} = \frac{{\sum }_{p = 1}^{N} ( {μ}_{ref , p} - {x}_{ref , p} ) {σ}_{ref , p}^{- 2}}{{\sum }_{p = 1}^{N} {σ}_{r e f , p}^{- 2}} = - 0.01072$$

Applying this exponent to the base of 10 we determine the scale factor for this sample/iteration as:

SF_{Overall}=10^{-0.010702}=0.9756

Similar to the procedure of SSAN, this intermediate scale factor would be applied to the measurements from sample 4 and the process would be repeated for the successive iterations.

FIG. 8B illustrates the scale factors determined by the application of ANML to the data in FIGS. 4A-4B over multiple iterations. The differences in normalized sample measurements between the first iteration and after convergence is quite distinct for those samples requiring more than 1 iteration. These additional iterations show benefits in data generated with an aptamer-based proteomic assay, which will be described further in the examples section. As shown in FIG. 8B, these scale factors differ from those determined by SSAN (FIG. 5E). These differences are due to the weighted population variance for each analyte, which helps balance the scale factor calculation for those analytes in which reference population variance is large.

FIG. 8C illustrates the normalized analyte levels resulting from the application of ANML to the data in FIGS. 4A-4B over multiple iterations. As shown in FIG. 8C, the normalized analyte levels differ from those determined by SSAN (FIG. 5B).

Another type of adaptive normalization that can be performed using the disclosed techniques is Population Adaptive Normalization (PAN). PAN can be utilized when the one or more samples comprise a plurality of samples and the one or more analyte levels corresponding to the one or more analytes comprise a plurality of analyte levels corresponding to each analyte.

When performing adaptive normalization using PAN, the distance between each analyte level in the one or more analyte levels and a corresponding reference distribution of that analyte in a reference data set is determined by determining a Student's T-test, Kolmogorov-Smirnov test, or a Cohen's D statistic between the plurality of analyte levels corresponding to each analyte and the corresponding reference distribution of each analyte in the reference data set.

For PAN, clinical data is treated as a group in order to censor analytes that are significantly different from the population reference data. PAN can be used when a group of samples is identified from having a subset of similar attributes such as being collected from the same testing site under certain collection conditions, or the group of samples may have a clinical distinction (disease state) that is distinct from the reference distributions.

The power of population normalization schemes is the ability to compare many measurements of the same analyte against the reference distribution. The general procedure of normalization is similar to the above-described adaptive normalization methods and again starts of an initial comparison of each analyte measurement against the reference distribution.

As explained above, multiple statistical tests can be used to determine statistical differences between analyte measurements from the test data and the reference distribution including Student's T-tests, Kolmogorov-Smirnov test, etc.

The following example utilizes the Cohen's D statistic for distance measurement, which a measurement of effect size between two distributions and is very similar to the M-distance calculation discussed previously:

$${D}_{p} = \frac{( {μ}_{p} - )}{\sqrt{{σ}_{ref , p}^{2} + {σ}_{x , p}^{2}}}$$

Where Dₚ is the Cohen's D statistic, μₚ is the reference distribution median for particular analyte, [symbol] is the clinical data (sample) median across all samples, and \sqrt{σ_{ref,p}²+σ_{x,p}²} is the pooled standard deviation (or median absolution deviation). As shown above, Cohen's D is defined as the difference between the reference distribution median and clinical data median over a pooled standard deviation (or median absolution deviation).

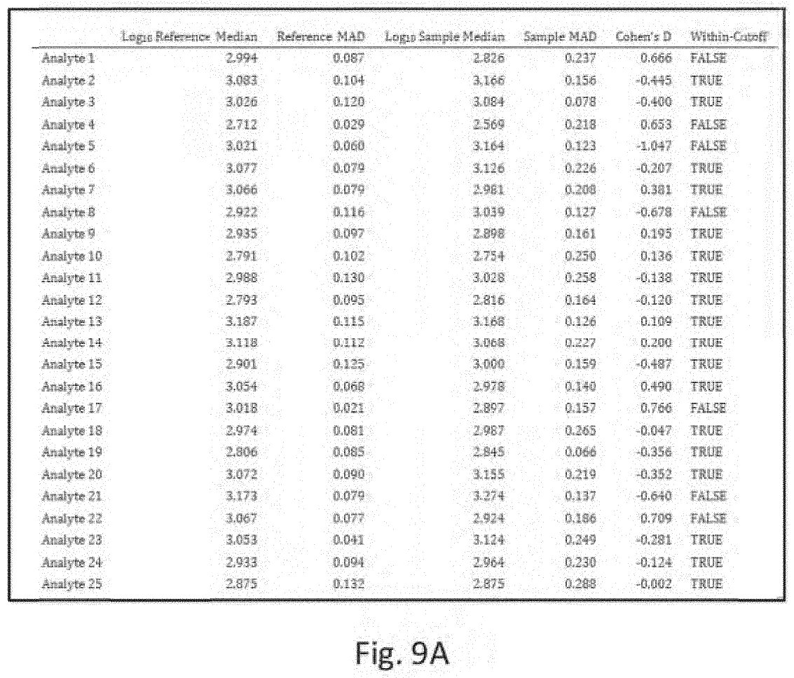

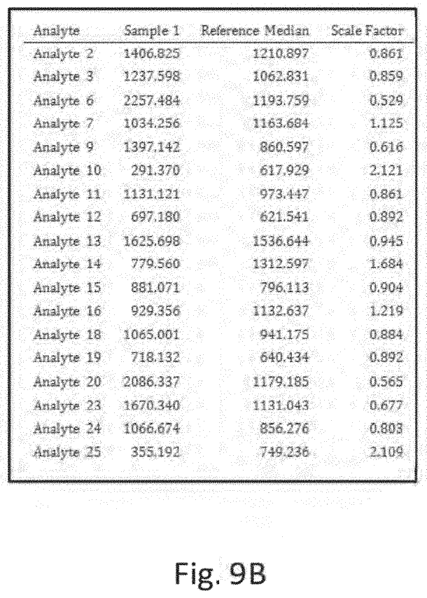

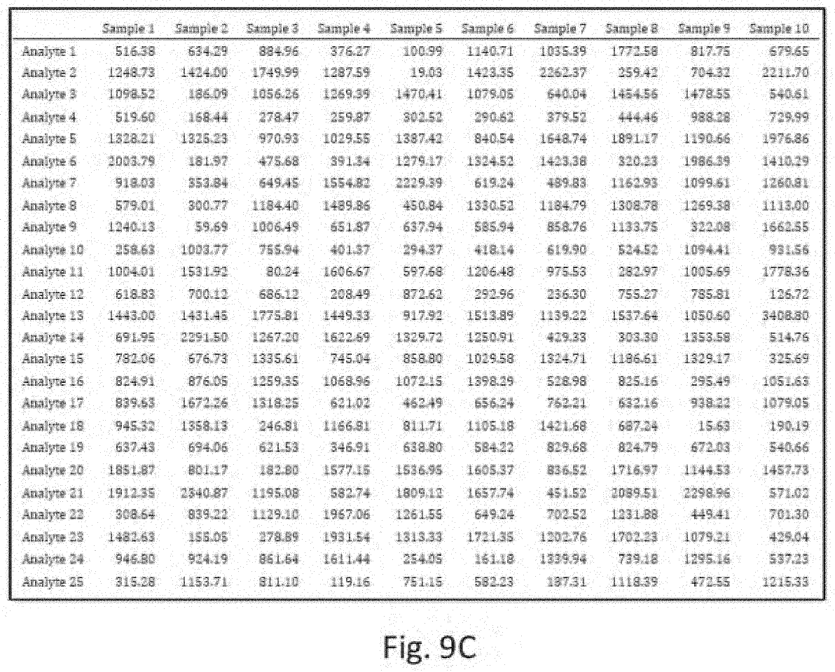

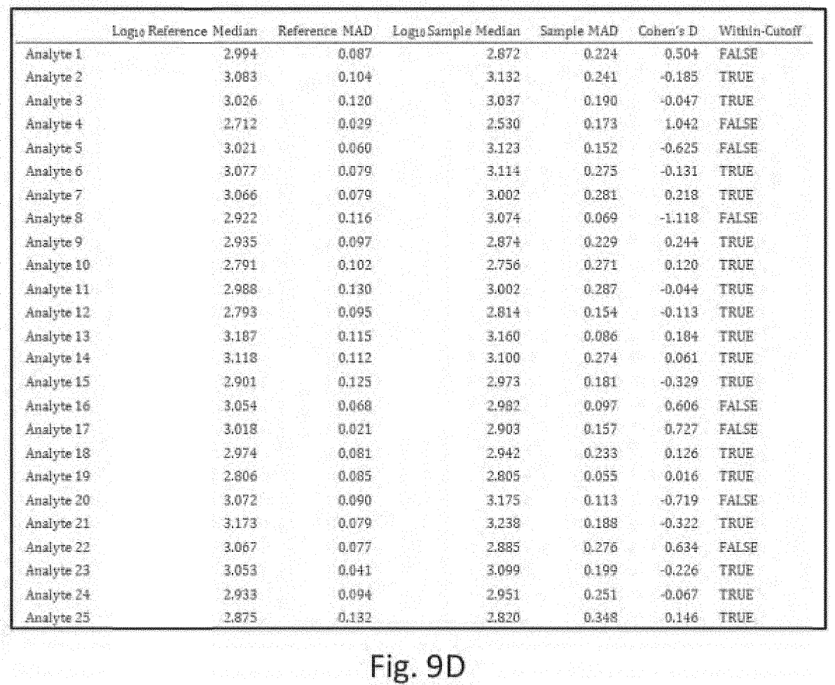

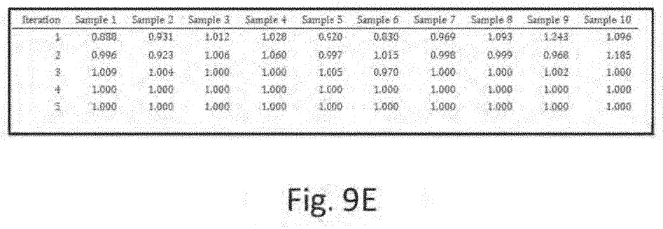

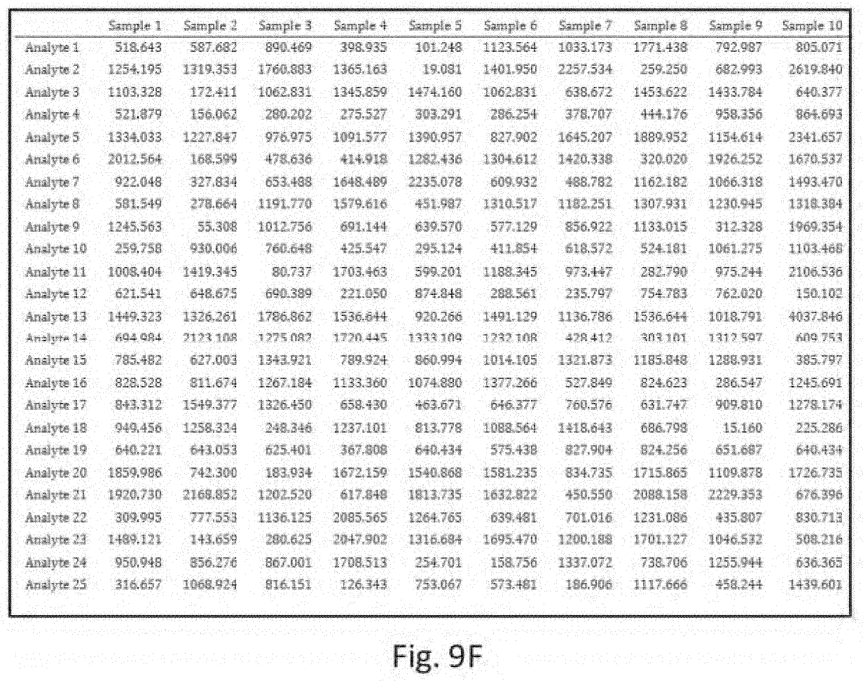

FIGS. 9A-9F illustrate the application of Population Adaptive Normalization to the data shown in FIGS. 4A-4B according to an exemplary embodiment. For the reference data shown in FIG. 4A and clinical data shown in FIG. 4B, 25 Cohen's D statistics are calculated, one corresponding to each analyte. FIG. 9A illustrates the Cohen's D statistic for each analyte across all samples. This calculation can be done in log 10 transformed space to enhance normality for analyte measurements.

In an exemplary embodiment, the predetermined distance threshold used to determine if an analyte is to be included in the scale factor determination process is a Cohen's D of |0.5|. Analytes outside of this window will be excluded from the calculation of scale factor. As shown in FIG. 9A, this results in analytes 1, 4, 5, 8, 17, 21, and 22 being excluded from the scale factor calculation.

FIG. 9B illustrates the scale factors calculated for each analyte across samples. A difference between population adaptive normalization (PAN) and the previously discussed normalization methods is that in PAN each sample will include/exclude the same analytes during scale factor calculation. In PAN, the scale factor for all samples will be determined on the basis of the remaining analytes. In this example, the scale factor can be given by the median or the mean of the analyte scale factors of the remaining analytes. Similar to the above-described adaptive normalization methods, the scale factor can be determined as a mean or median of the individual analyte scale factors. If the median is used, then the scale factor for the data shown in FIG. 9B is 0.8876.

This scale factor is multiple with the data values shown in FIG. 4B to generate normalized data values, as shown in FIG. 9C. FIG. 9D illustrates the results of the second iteration of the scale factor determination process, including the Cohen's D value for each analyte and the Within-Cutoff value for each analyte.

For this iteration, analytes 1, 4, 5, 8, 16, 17, 20, and 22 are to be excluded from the scale factor determination process. In addition to the analytes excluded in the first iteration, the second iteration additionally excludes analyte 16 from the calculation of scale factors. The above-described steps are then repeated to removing the additional analyte from scale factor calculation for each sample.

Convergence of the adaptive normalization (a change in scale factor less than a predefined threshold) occurs when the analytes removed from the i^{th} iteration are identical to the (i−1)^{th} iteration and scale factors for all samples have converged. In this example, convergence requires five iterations. FIG. 9E illustrates the scale factors for each of the samples at each of the five iterations. Additionally, FIG. 9F illustrates the normalized analyte level data after convergence has occurred and all scale factors have been applied.

The systems and methods described herein implement an adaptive normalization process which performs outlier detection to identify any outlier analyte levels and exclude said outliers from the scale factor determination, while including the outliers in the scaling aspect of the normalization.

The features of computing a scale factor and applying the scale factor are also described in greater detail with respect to the previous figures. Additionally, the removal of outlier analyte levels in the one or more analyte levels by performing outlier analysis can be implemented as described with respect to FIGS. 1-3.

The outlier analysis method described in those figures and the corresponding sections of the specification is a distance based outlier analysis that filters analyte levels based upon a predetermined distance threshold from a corresponding reference distribution.

However, other forms of outlier analysis can also be utilized to identify outlier analyte levels. For example, a density based outlier analysis such as the Local Outlier Factor (“LOF”) can be utilized. LOF is based on local density of data points in the distribution. The locality of each point is given by k nearest neighbors, whose distance is used to estimate the density. By comparing the local density of an object to the local densities of its neighbors, regions of similar density can be identified, as well as points that have a lower density than their neighbors. These are considered to be outliers.

Density-based outlier detection is performed by evaluating distance from a given node to its K Nearest Neighbors (“K-NN”). The K-NN method computes a Euclidean distance matrix for all clusters in the cluster system and then evaluates local reachability distance from the center of each cluster to its K nearest neighbors. Based on the said distance matrix local reachability distance, density is computed for each cluster and the Local Outlier Factor (“LOF”) for each data point is determined. Data points with large LOF value are considered as the outlier candidates. In this case, the LOF can be computed for each analyte level in the sample with respect to its reference distribution.

The step of normalizing the one or more analyte levels over one or more iterations can include performing additional iterations until a change in the scale factor between consecutive iterations is less than or equal to a predetermined change threshold or until a quantity of the one or more iterations exceeds a maximum iteration value, as discussed previously with respect to FIG. 1.

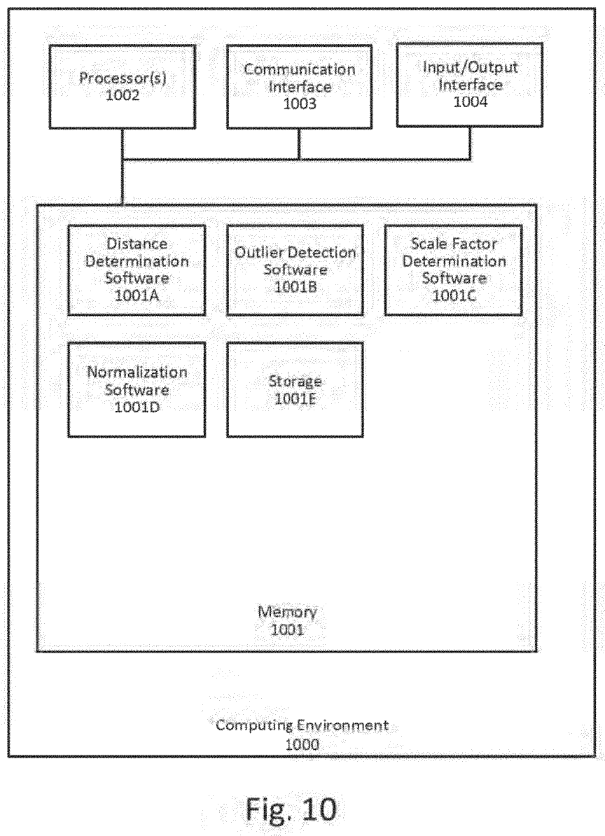

FIG. 10 illustrates a specialized computing environment for adaptive normalization of analyte levels according to an exemplary embodiment. Computing environment 1000 includes a memory 1001 that is a non-transitory computer-readable medium and can be volatile memory (e.g., registers, cache, RAM), non-volatile memory (e.g., ROM, EEPROM, flash memory, etc.), or some combination of the two.

As shown in FIG. 10, memory 1001 _{[MW2]} stores distance determination software 1001A for determining statistical/mathematical distances between analyte levels and their corresponding reference distributions, outlier detection software 1001B for identifying analyte levels that are outside the predefined distance threshold, scale factor determination software 1001C for determining analyte scale factors and overall scale factors, normalization software 1001D for applying the adaptive normalization techniques described herein to a data set.

Memory 1001 additionally includes a storage 1001 that can be used to store the reference data distributions, statistical measures on the reference data, variables such as the scale factor and Boolean data structures, intermediate data values or variables resulting from each iteration of the adaptive normalization process.

All of the software stored within memory 1001 can be stored as computer-readable instructions, that when executed by one or more processors 1002, cause the processors to perform the functionality described herein.

Processor(s) 1002 execute computer-executable instructions and can be a real or virtual processor. In a multi-processing system, multiple processors or multicore processors can be used to execute computer-executable instructions to increase processing power and/or to execute certain software in parallel.

The computing environment additionally includes a communication interface 503, such as a network interface, which is used to monitor network communications, communicate with devices, applications, or processes on a computer network or computing system, collect data from devices on the network, and actions on network communications within the computer network or on data stored in databases of the computer network. The communication interface conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

Computing environment 1000 further includes input and output interfaces 1004 that allow users (such as system administrators) to provide input to the system and display or otherwise transmit information for display to users. For example, the input/output interface 1004 can be used to configure settings and thresholds, load data sets, and view results.

An interconnection mechanism (shown as a solid line in FIG. 10), such as a bus, controller, or network interconnects the components of the computing environment 1000.

Input and output interfaces 1004 can be coupled to input and output devices. The input device(s) can be a touch input device such as a keyboard, mouse, pen, trackball, touch screen, or game controller, a voice input device, a scanning device, a digital camera, remote control, or another device that provides input to the computing environment. The output device(s) can be a display, television, monitor, printer, speaker, or another device that provides output from the computing environment 1000. Displays can include a graphical user interface (GUI) that presents options to users such as system administrators for configuring the adaptive normalization process.

The computing environment 1000 can additionally utilize a removable or non-removable storage, such as magnetic disks, magnetic tapes or cassettes, CD-ROMs, CD-RWs, DVDs, USB drives, or any other medium which can be used to store information and which can be accessed within the computing environment 1000.

The computing environment 1000 can be a set-top box, personal computer, a client device, a database or databases, or one or more servers, for example a farm of networked servers, a clustered server environment, or a cloud network of computing devices and/or distributed databases.

As used herein, “nucleic acid ligand,” “aptamer,” “SOMAmer,” and “clone” are used interchangeably to refer to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule. In one embodiment, the action is specific binding affinity for a target molecule, such target molecule being a three dimensional chemical structure other than a polynucleotide that binds to the aptamer through a mechanism which is independent of Watson/Crick base pairing or triple helix formation, wherein the aptamer is not a nucleic acid having the known physiological function of being bound by the target molecule. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target generally with a much higher degree of affinity than it binds to other, non-target, components in a mixture or sample. An “aptamer,” “SOMAmer,” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In some embodiments, the aptamers are prepared using a SELEX process as described herein, or known in the art. As used herein, a “SOMAmer” or Slow Off-Rate Modified Aptamer refers to an aptamer having improved off-rate characteristics. SOMAmers can be generated using the improved SELEX methods described in U.S. Pat. No. 7,947,447, entitled “Method for Generating Aptamers with Improved Off-Rates,” the disclosure of which is hereby incorporated by reference in its entirety.

Greater detail regarding aptamer-base proteomic assays are described, in U.S. Pat. Nos. 7,855,054, 7,964,356 and 8,945,830, U.S. patent application Ser. No. 14/569,241, and PCT Application PCT/US2013/044792, the disclosures of which are hereby incorporated by reference in their entirety.

### EXAMPLES

Improved Precision

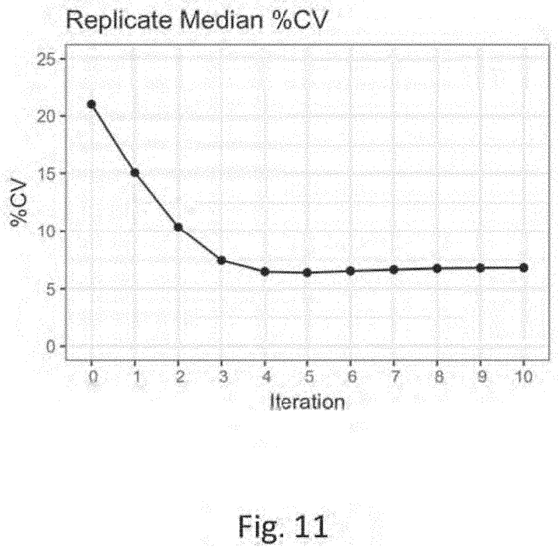

FIG. 11 illustrates median coefficient of variation across all aptamer-based proteomic assay measurements for 38 technical replicates.

Applicant took 38 technical replicates from 13 aptamer based proteomic assay runs (Quality Control (QC)samples) and calculated coefficient of variation (CV), defined as the standard deviation of measurements over the mean/median of measurements, for each analyte across the aptamer-based proteomic assay menu. Using ANML, Applicant normalized each sample while controlling the maximum number of iterations each sample would be allowed under the normalization process.

The median CVs for the replicates show reduced CV as the maximum number of allowable iterations increases indicating increased precision as replicates are allowed to converge.

Improved Biomarker Discrimination

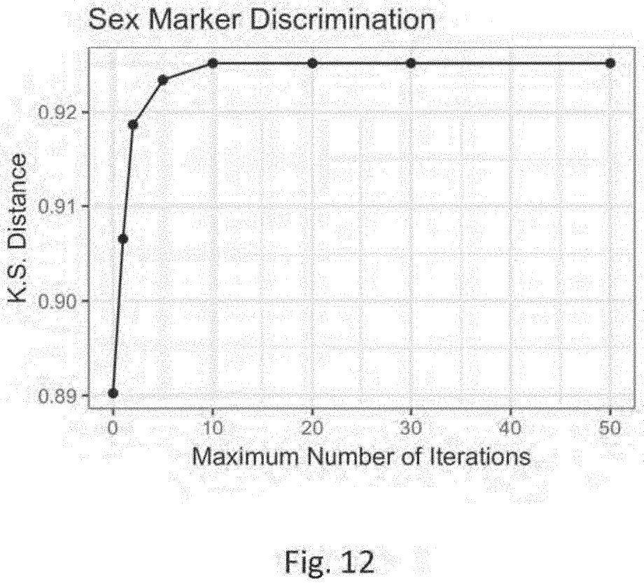

FIG. 12 illustrates the Kolmogorov-Smirnov statistic against a gender specific biomarker for samples with respect to maximum allowable iterations.

Applicant looked at the discriminatory power for a gender specific biomarker known in the aptamer-based proteomic assay menu. Applicant calculated a Kolmogorov-Smirnov (K.S.) test to quantify the distance between the empirical distribution functions of 569 female and 460 male samples to quantify the extent of separation between this analyte shows between male/female samples where a K.S. distance of 1 implies complete separation of distribution (good discriminatory properties) and 0 implies complete overlap of the distributions (poor discriminatory properties). As in the example above, Applicant limited the number of iterations each sample could run through before calculating the K.S. distance of the groups.

This data shows that the discriminatory characteristics of the biomarker for male/female gender determination are increased as samples are allowed to converge in the iterative normalization process.

Application of Anvil on QC Samples

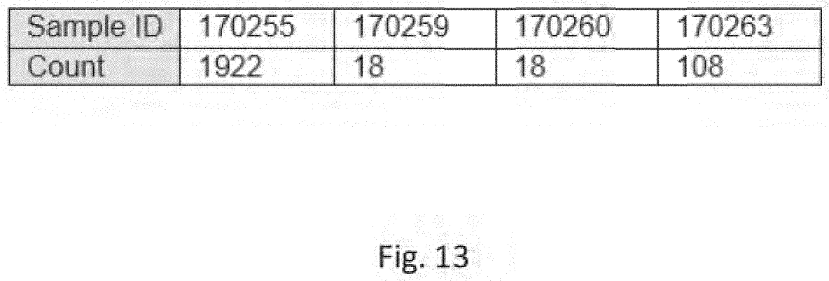

662 runs (BI, in Boulder) with 2066 QC samples. These replicates comprise 4 different QC lots. FIG. 13 illustrates the number of QC samples by SampleID for plasma and serum used in analysis.

A new version of the normalization population reference was generated (to make it consistent with the ANML and generate estimates to the reference SDs). The data described above was hybridization normalized and calibrated as per standard procedures for V4 normalization. At that point, it was median normalized to both the original and the new population reference (shows differences due to changes in the median values of reference) and using ANML (shows differences due to both the adaptive and maximum likelihood changes in normalization to a population reference.)

Normalization Scale Factors

A first comparison to make is to look at the scale factors concordances between different normalization references/methods. If there are only slight differences, then good concordance in all other metrics is to be expected. FIG. 1 shows scale factors for QC samples in plasma and serum; which show good concordance between For QC_1710255 (for which we have, by far, the largest number of replicates), for the most part, there is no large difference (the dashed lines represent a difference of 0.1 in scale factors; so differences are mostly below 0.05.)

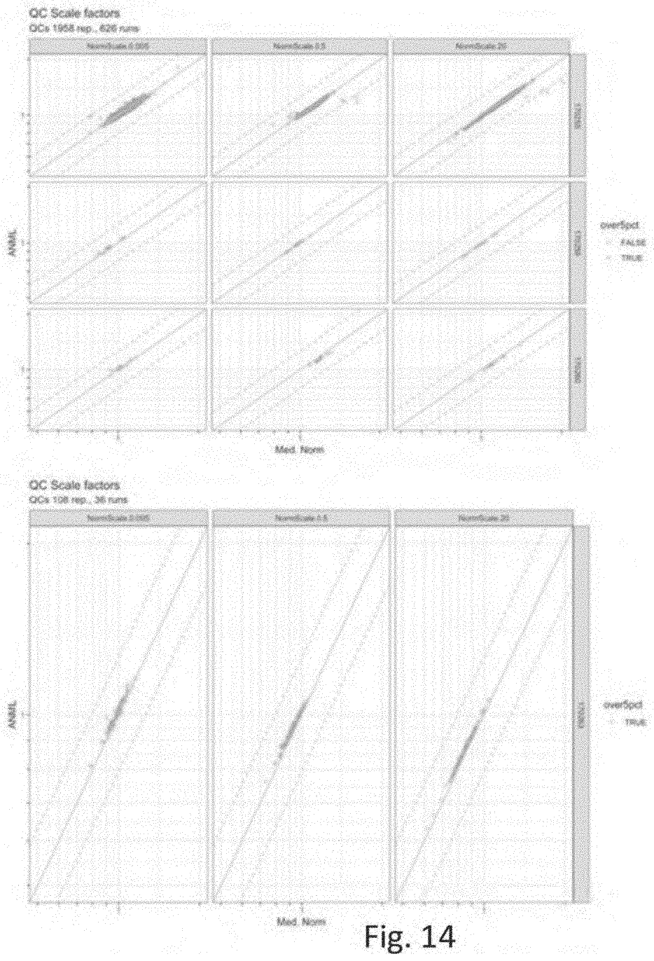

FIG. 14 illustrates the concordance of QC sample scale factors using median normalization and ANML. Solid line indicates identity, dashed lines indicate difference of 0.1 above/below identity.

CV's

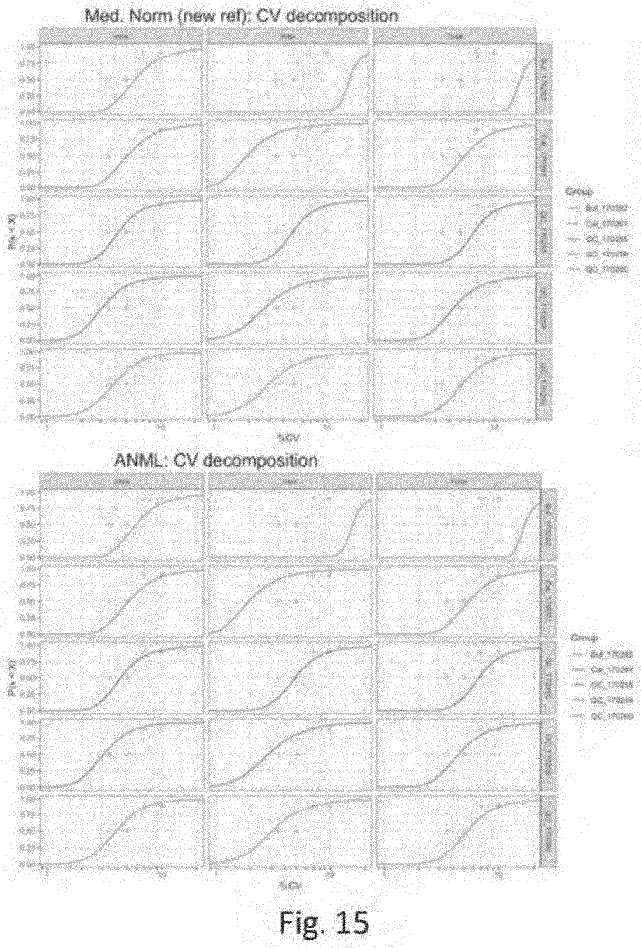

We then computed the CV decomposition for control samples in plasma and serum samples in median normalization and ANML. FIG. 15 illustrates CV Decomposition for control samples using median normalization and ANML. Lines indicate empirical cumulative distribution function of CV for each control samples within a plate (intra) between plates (inter) and total.

There is little (if any) discernable difference between the two normalization strategies indicating that ANVIL does not change control sample reproducibility.

QC Ratios to Reference

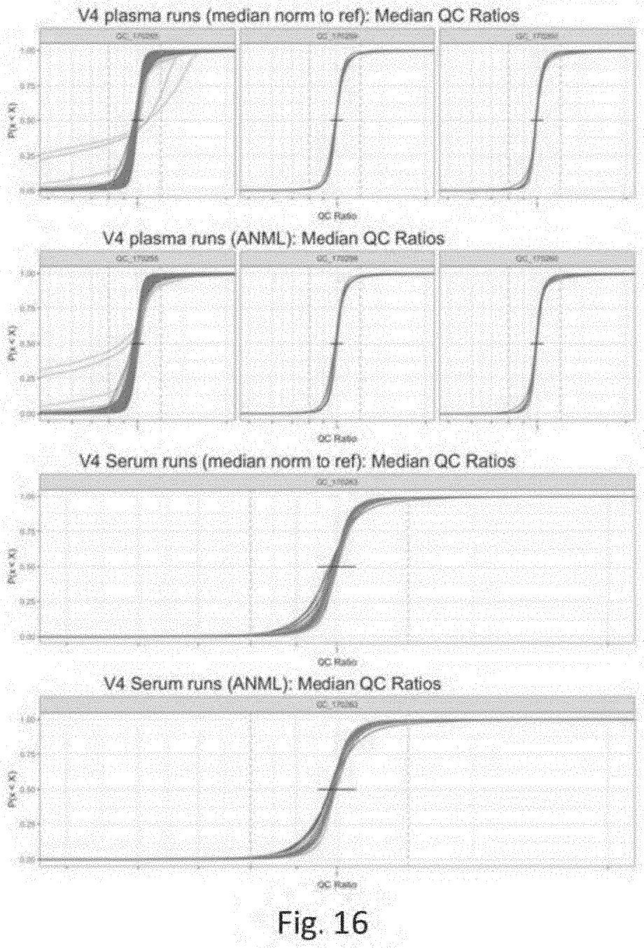

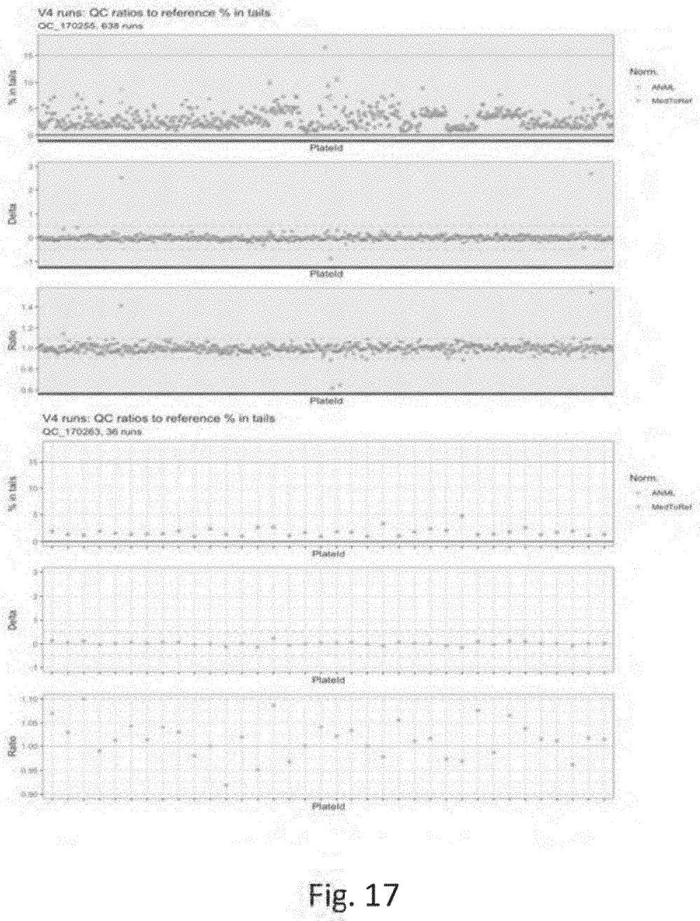

After ANML, we compute references for each of the QC lots, and use these reference values to compare to the median QC value in each run. Empirical cumulative distribution functions for QC samples in plasma and serum. FIG. 16 illustrates median QC ratios using median normalization and ANVIL. Each line indicates an individual plate. These ratios distributions show that when we had a “good” distribution, then it did not change much when using ANML. On the other hand, a couple of abnormal distributions (plasma, in light blue) get somewhat better under ANVIL. It does not seem like the tails are much affected, but to make sure we plot below the % in tail for both methods, as well as their differences and ratios. FIG. 17 illustrates QC ratios in tails using median normalization and ANVIL. Each dot indicates an individual plate, the yellow line indicates plate failure criteria and he dotted lines in the Delta plot are at +−0.5%, while the ones at the ratio plot at 0.9, 1.1.

We see that there is no change in failures (the only plotted run that was over 15% in tails remains there; the abnormal ones that were not plotted remain abnormal.) Moreover, differences in tails are well below 0.5% for almost all runs.

Application of ANML on Datasets

We compared the effects of ANML against SSAN on clinical (Covance) and experimental (time-to-spin) datasets using consistent Mahalanobis distance cutoff of 2.0 for analyte exclusion during normalization.

Time-to-Spin

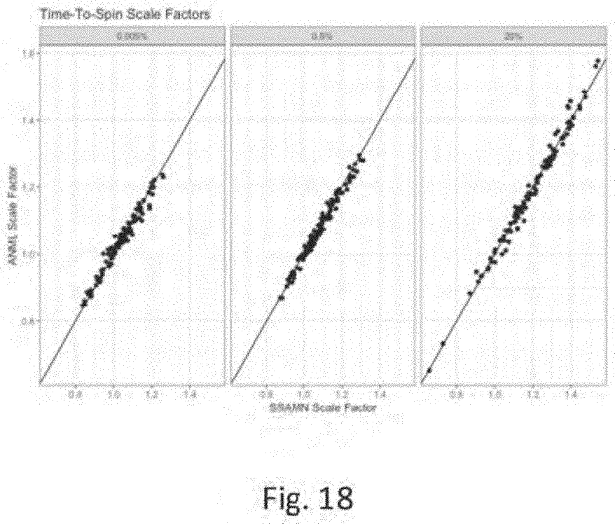

The time-to-spin experiment used 18 individuals each of 6 K2EDTA-Plasma blood collection tubes that were left to sit for 0, 0.5, 1.5, 3, 9, and 24 hours before processing. Several thousand analytes show signal changes a function of processing time, the same analytes that show similar movement with clinical samples with uncontrolled or with processing protocols not in-line with SomaLogic's collection protocol. We compared the scale factors from SSAN against ALMN. FIG. 18 illustrates scale factor concordance in time-to-spin samples using SSAN and ANML. Each dot indicates an individual sample. There is very good agreement between the two methods.

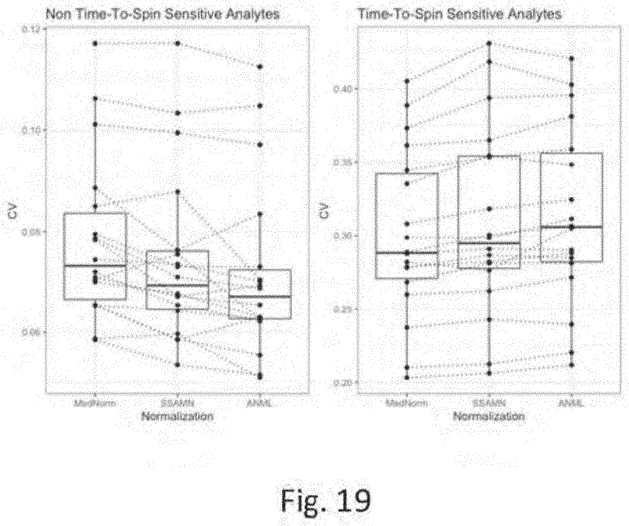

This dataset is unique in that multiple measurements of the same individual under increasingly detrimental sample quality. While many analyte signals are affected by time-to-spin there are many thousands that are unaffected as well. The reproducibility of these measurements across increasing time-to-spin can be quantified across multiple normalization schemes; standard median normalization, single sample adaptive median normalization, and adaptive normalization by maximum likelihood. We calculated CV's for each of the 18 donors across time-to-spin, separating the analytes by their sensitivity to time-to-spin. FIG. 19 illustrates median analyte CV's across 18 donors in time-to-spin under varying normalization schemes. Each dot indicates 1 individual joined by dashed lines across varying normalization

The expectation for analytes that do not show sensitivity to time-to-spin should be high reproducibility for each donor across the 6 conditions and thus the adaptive normalization strategy should lower CVs.

ANML shows improved CVs against both standard median normalization and SSAN indicating that this normalization procedure is increasing reproducibility against detrimental sample handling artifacts. Conversely, analytes affected by time-to-spin (FIG. 19) which are amplified over the 6 time-to-spin conditions. This is consistent with previous observations that an adaptive normalization scheme will enhance true biological effects. In this case sample handling artifacts are magnified, however in other cases such as chronic kidney disease where many analytes are affected, we expect a similar broadening of effect sizes for those effected analytes.

Covance

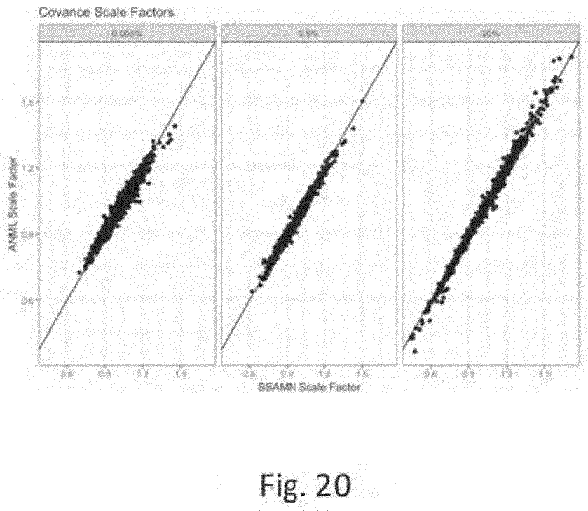

We next tested ANML on Covance plasma samples which were used to derive the population reference. The comparison of scale factors obtained using the single sample adaptive schemes are presented by dilution group in FIG. 20. FIG. 20 illustrates a concordance plot between scale factors from Covance (plasma) using SSAN and ANML. Each dot indicates an individual, solid line indicates identity. Very good agreement is again obtained between the two methods.

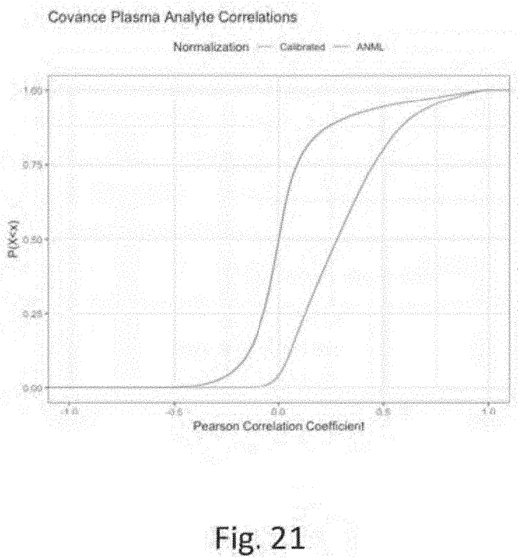

A goal of normalization is to remove correlated noise that results during the aptamer-based proteomic assay. FIG. 21 shows the distribution of all pairwise analyte correlations for Covance samples before and after ANML. The red curve shows the correlation structure of calibrated data which shows a distinct positive correlation bias with little to no negative correlations between analytes. After normalization this distribution is re-centered with distinct populations of positive and negative correlating analytes.

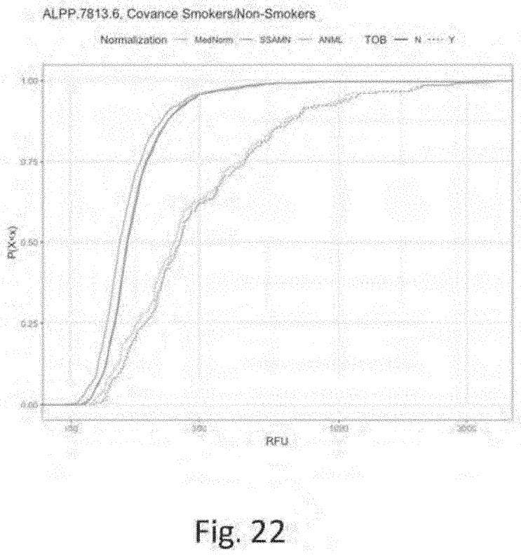

We next looked how ANML compared to SSAN on insight generation and testing using Covance smoking status. FIG. 22 illustrates a comparison of distributions obtained from data normalized through several methods. The distributions for tobacco users (dotted lines) and nonusers (solid lines) for these two analytes are virtually identical between ANML and SSAN. The distribution of alkaline phosphatase shown in FIG. 22 is a top predictor of smoking use status, which shows good discrimination under ANML.

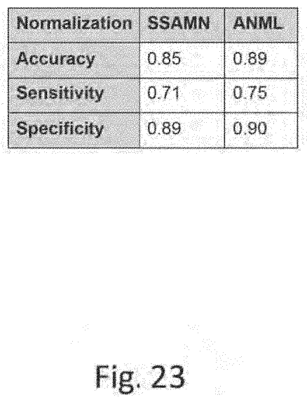

We trained a logistic regression classifier for predicting smoking status using a complexity of 10 analytes under SAMN normalized data and ANML normalized data using an 80/20 train/test split. A summary of performance metrics for each normalization is shown in FIG. 23, which illustrates metrics for smoking logic-regression classifier model for hold-out test set using data normalized with SSAN and ANML. Under ANML we see no loss, and potentially a small gain, in performance for smoking prediction.

Adaptive normalization by maximum likelihood uses information of the underlying analyte distribution to normalize single samples. The adaptive scheme guards against the influence of analytes with large pre-analytic variations from biasing signals from unaffected analytes. The high concordance of scale factors between ANML and single sample normalization shows that while small adjustments are being made, they can influence reproducibility and model performance. Furthermore, data from control samples show no change in plate failures or reproducibility of QC and calibrator samples.

Application of Pan on Datasets

The analysis begins with data that was hybridization normalized and calibrated internally. In all the following studies, unless otherwise noted, the adaptive normalization method uses Student's t-test for detecting differences in the defined groups along with the BH multiple test correction. Typically, the normalization is repeated with different cutoff values to examine the behavior. In all cases, adaptive normalization is compared to the standard median normalization scheme.

Covance

Covance collected plasma and serum samples from healthy individuals across five different collection sites: San Diego, Honolulu, Portland, Boise, and Austin/Dallas. Only one sample from the Texas site was assayed and so was removed from this analysis. The 167 Covance samples for each matrix were run on the aptamer-based proteomic assay (V3 assay; 5k menu). The directed groups here are defined by the first four collection sites.

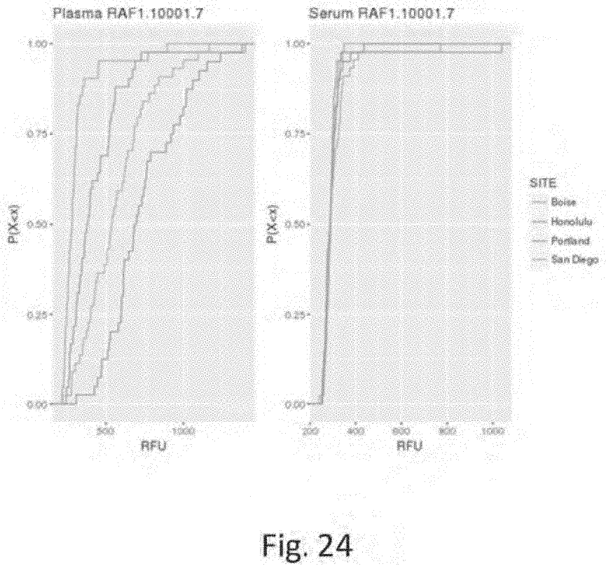

The number of analytes removed in Covance plasma samples using adaptive normalization is ˜2500 or half the analyte menu, whereas, measurements for Covance serum samples do not show any significant amount of site biases and less than 200 analytes were removed. The empirical cumulative distribution functions (cdfs) by collection site for analyte measurement c-RAF illustrates the site bias observed for plasma measurements and lack of such bias in serum. FIG. 24 illustrates Empirical CDFs for c-Raf measurements in plasma and serum samples colored by collection site. Notable differences in plasma sample distribution (left) are collapsed in serum samples (right). Adaptive normalization only removes analytes within a study that are deemed problematic by statistical tests, so the plasma and serum normalization for Covance are sensibly tailored to the observed differences.

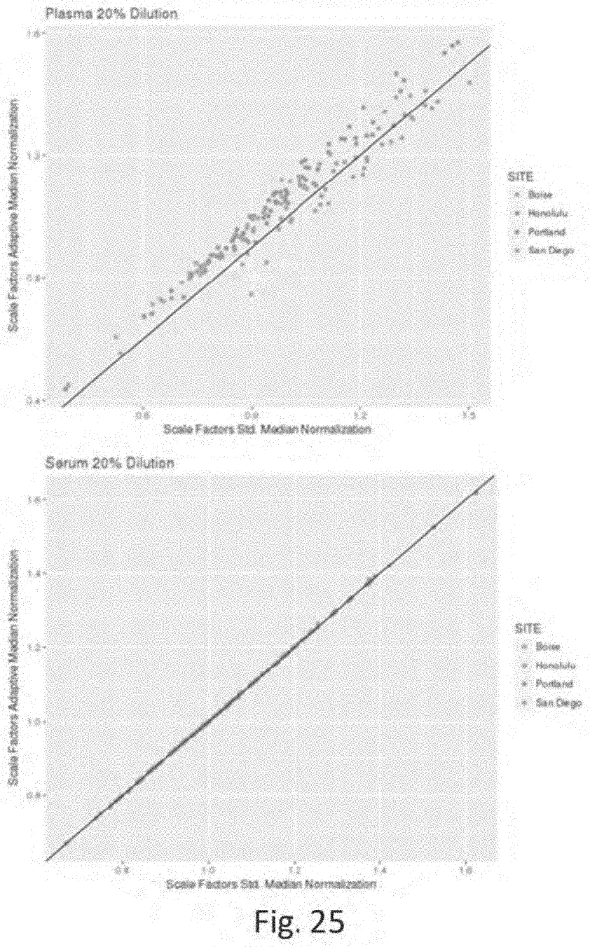

A core assumption with median normalization is that the clinical outcome (or in this case collection site) affects a relatively small number of analytes, say <5%, to avoid introducing biases in analyte signals. This assumption holds well for the Covance serum measurements and is clearly not valid for the Covance plasma measurements. Comparison of median normalization scale factors from our standard procedure with that of adaptive normalization reveals that for serum, adaptive normalization faithfully reproduces scale factors for the standard scheme. However, for plasma, many analyte measurements will have site-dependent biases introduced by using the standard normalization procedure. FIG. 25 illustrates concordance plots of scale factors using standard median normalization vs. adaptive median normalization in plasma (top) and serum (bottom). In plasma, several thousand analytes show significant site biases which is accounted for and corrected using the adaptive scheme. In serum, <200 analytes show significant site biases resulting in little to no change in scale factors between the two normalization schemes. Individual points represent scale factors for each sample colored by collection site. Black line indicates identity.

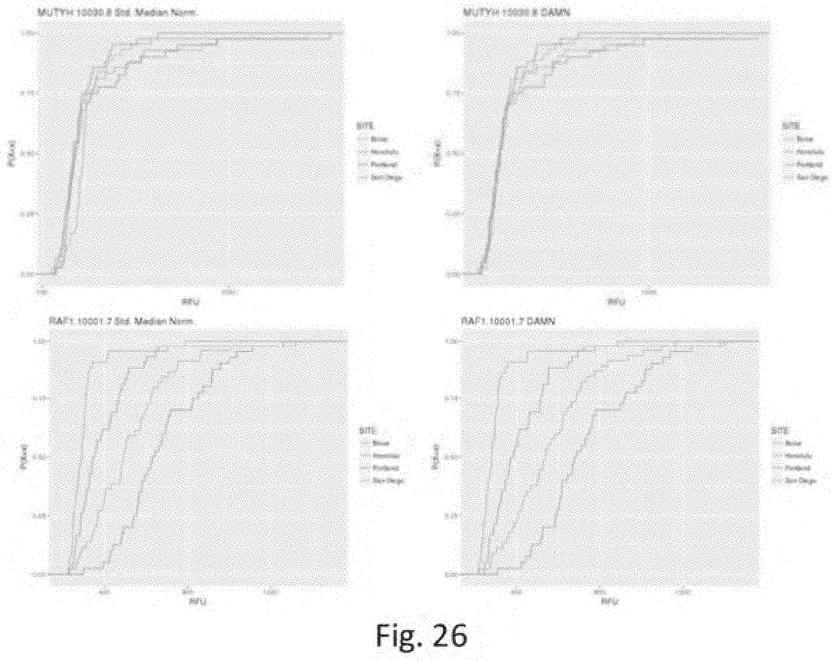

For example, consider analytes that are not signaling differently among the four sites in plasma. Due to the large number of other analytes that are signaling higher in Honolulu, Portland and San Diego samples, the measurements for these analytes after standard median normalization will be inflated for the Boise site while simultaneously being deflated for the remaining three sites, introducing a clear artifact in the data. This is observed in the plasma scale factors for Boise samples appearing below the diagonal while the rest appear above the diagonal in FIG. 25. To illustrate the bias that misapplication of standard median normalization can induce, CDFs by site for an analyte that is not affected by the site differences are displayed in FIG. 26 for the standard normalization scheme and adaptive normalization. The adaptive normalization performs well for guarding against introducing artifacts in the data during normalization due to collection site bias. For analytes that show strong site bias, adaptive normalization will preserve the differences while standard median normalization tends to dampen these differences, see c-RAF in FIG. 26. The median RFUs for all sites except Boise are higher in the adaptive normalization set compared to standard.

The Covance results illustrate two key features of the adaptive normalization algorithm, (1) for datasets with no collection site or biological bias, adaptive normalization faithfully reproduces the standard median normalization results, as illustrated for the serum measurements. For situations in which multiple sites or pre-analytical variation or other clinical covariates affect many analyte measurements, adaptive normalization will normalize the data correctly by removing the altered measurements during scale factor determination. Once a scale factor has been computed, the entire sample is scaled.

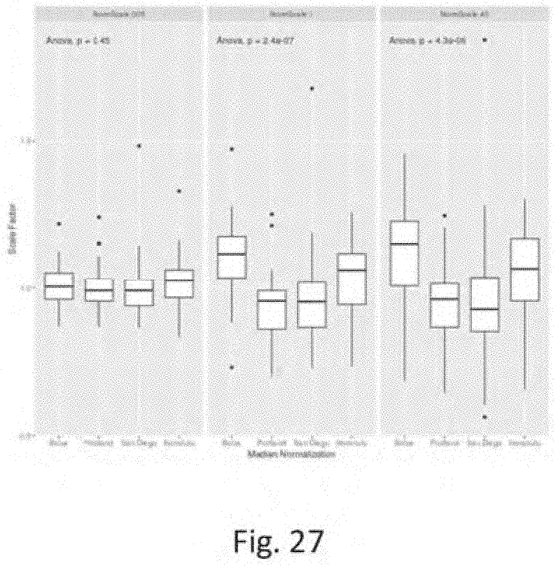

In practice, artifacts in median normalization can be detected by looking for bias in the set of scale factors produced during normalization. With standard median normalization, there are significant differences in scale factor distributions among the four collection sites—with Portland and San Diego more similar than Boise and Honolulu. FIG. 27 illustrates plasma sample median normalization scale factors by dilution and Covance collection site. The bias in scale factors by site is most evident for measurements in the 1% and 40% mix. A simple ANOVA test on the distribution of scale factors by site indicates statistically significant differences for the 1% and 40% dilution measurements with p-values of 2.4×10⁻⁷ and 4.3×10⁻⁶ while the measurements in the 0.005% dilution appear unbiased, with a p-value of 0.45. The ANOVA test for scale factor bias among the defined groups for adaptive normalization provide a key metric for assessing normalization without introduction of bias.

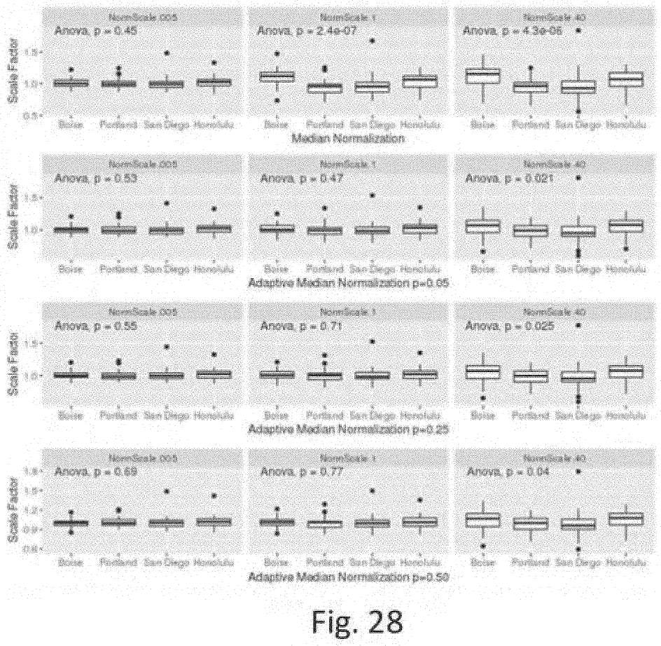

This is illustrated in FIG. 28 where the distributions of median normalization scale factors are shown for increasing stringency in adaptive normalization, from q-value cutoff of 0.0 (standard median normalization), 0.05, 0.25, and 0.5. At a 0.05 cutoff, 2557 (˜50%) of analytes were identified as showing variability with collection site. Increasing the cutoff to 0.25 and 0.5 identifies 3479 and 4133 analytes. However, the extent to which increasing the cutoff removes site specific difference in median scale factors is negligible. Measurements in the 1% dilution no longer show site specific differences in scale factors while site bias in the 40% dilution have been reduced significantly, by four logs in q-value, and the 0.005% distribution was unchanged and unbiased to begin with.

Sample Handling/Time-to-Spin

Samples collected from 18 individuals in-house with multiple tubes per individual sat before spinning for 0, 0.5, 1.5, 3, 9, and 24 hours at room temperature. Samples were run using standard aptamer-based proteomic assay.

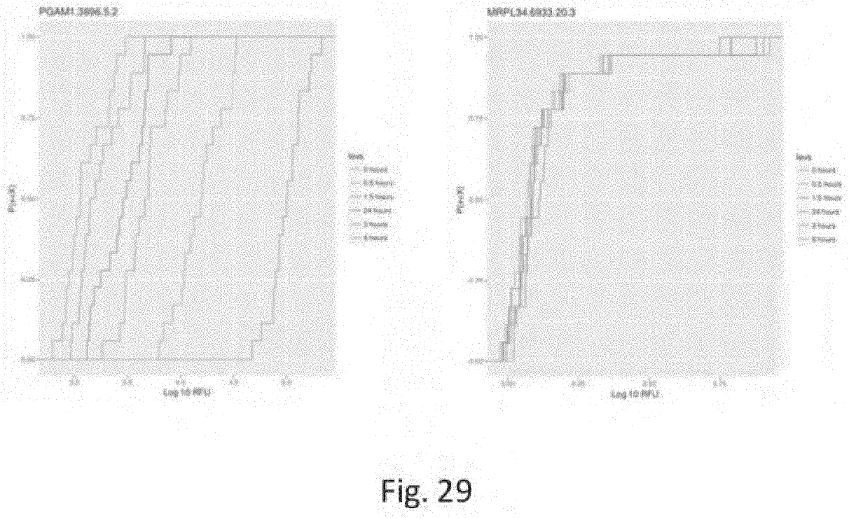

Certain analyte's signals are dramatically affected by sample handling artifacts. For plasma samples, specifically, the duration that samples are left to sit before spinning can increase signal by over ten-fold over samples that are promptly processed. FIG. 29 shows typical behavior for an analyte which shows significant differences in RFU as a function of time-to-spin.

Many of the analytes that are seen to increase in signal with increasing time-to-spin have been identified as analytes that are dependent on platelet activation (data not shown). Using measurements for analytes like this within median normalization introduces dramatic artifacts in the process, and entire samples that are unaffected by the spin time can be negatively altered. Conversely, FIG. 29 also shows a sample analyte insensitive to time-to-spin whose measurements may become distorted by including analytes in the normalization procedure that are affected by spin time. It is critical to remove any measurement that is aberrant—for whatever reason—from the normalization procedure to assure the integrity of the remaining measurements.

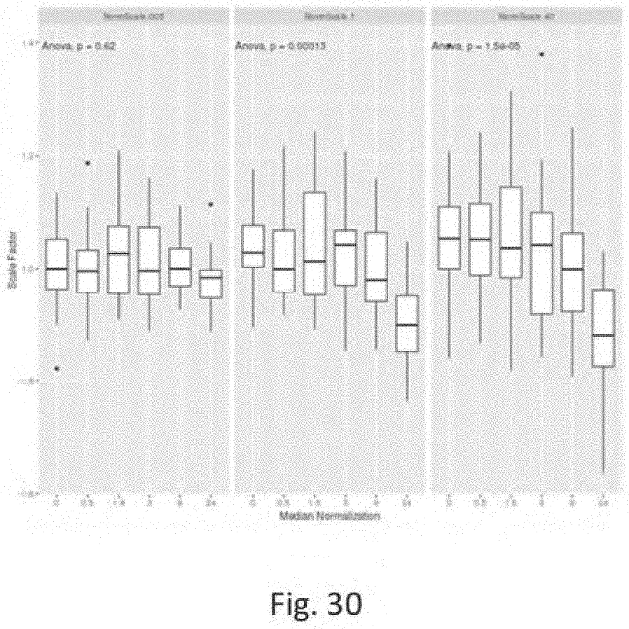

Standard median normalization across this time-to-spin data set will lead to significant, systematic differences in median normalization scale factors across the time-to-spin groups. FIG. 30 illustrates median normalization scale factors by dilution with respect to time-to-spin. Samples left for long periods of time before spinning result in higher RFU values, leading to lower median scale factors.

The scale factors for the 0.005% dilution are much less affected by spin time than the 1% and 40% dilutions. This is probably due to two distinctly different reasons. The first is that the number of highly abundant circulating analytes that are also in platelets is relatively small, therefore fewer plasma analytes in the 0.005% dilution are affected by platelet activation. In addition, extreme processing times may lead to cell death and lysis in the samples, releasing nuclear proteins that are quite basic (histones, for example) and increase the Non-Specific Binding (NSB) as evidenced by signals on negative controls. Due to the large dilution, the effect of NSB is not observed in 0.005% dilution. Median normalization scale factors for the 1% and 40% dilution exhibit quite strong bias with spin times. Due to the predominately increase in signal with increasing spin time, short spin time samples have higher scale factors than one—signals are increased by median normalization—and samples with longer spin times have scale factors lower than one—signals are reduced. Such observed bias in the normalization scale factors gives rise to bias in the measurements for those analytes unaffected by spin time, similar to that illustrated above in the Covance samples.

Many analytes are affected by platelet activation in plasma samples, so these data represent an extreme test of the adaptive normalization method since both the number of affected analytes and the magnitude of the effect size is quite large. We tested if our adaptive normalization procedure could remove this inherent correlation between median normalization scale factors and the time-to-spin.

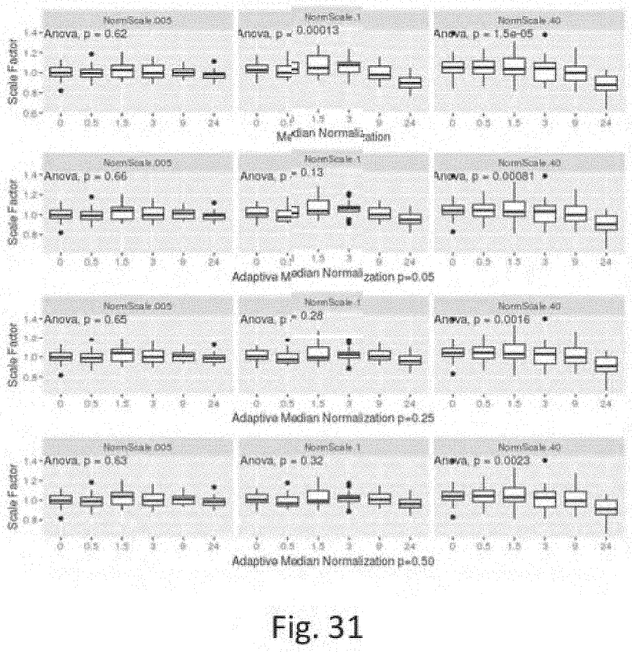

Adaptive normalization was run against the plasma time-to-spin samples using Kruskal-Wallis to test for significant differences, using BH to control for multiple comparisons. Bonferroni multiple comparisons correction was also used and generated similar results (not shown). At a cutoff of p=0.05, 1020, or 23%, of analytes were identified as showing significant changes with time-to-spin. Increasing the cutoff to 0.25 and 0.5 increases the number of significant analytes to 1344 and 1598, respectively. The effect of adaptive normalization on median normalization scale factors vs. time-to-spin is summarized in FIG. 31.

analytes within the 0.005% dilution were unbiased with the standard median normalization and their values were unaffected by adaptive normalization. While at all cutoff levels the variability in the scale factors with spin time for the 1% dilution is removed, there is still some residual bias in the 40% dilution, albeit it has been dramatically reduced. There is evidence to suggest that the residual bias may be due to NSB induced by platelet activation and/or cell lysis.

To summarize, using a fairly stringent cutoff of 0.25 for adaptive normalization does result in normalization across this sample set that decreases the bias observed in the standard normalization scheme but does not completely mitigate all artifacts. This may be due to NSB that is a confounding factor here and adaptive normalization removes this signal on average, resulting in the remaining bias in scale factors but potentially removing bias in analyte signals.

CKD/GFR (CL-13-069)

A final example of the usefulness of PBAN includes a dataset from a single site with presumably consistent collection but with quite large biological effects due to the underlying physiological condition of interest, Chronic Kidney Disease (CKD). The CKD study, comprising 357 plasma samples, was run on the aptamer-based proteomic assay (V3 assay; 1129-plex menu). Samples were collected along with Glomerular Filtration Rate (GFR) as a measure of kidney function where GFR ranges >90 mls/min/1.73 m² for healthy individuals. GFR was measured for each sample using iohexol either pre or post blood draw. We made no distinction in the analysis for pre/post iohexol treatment however paired samples were removed from analysis.

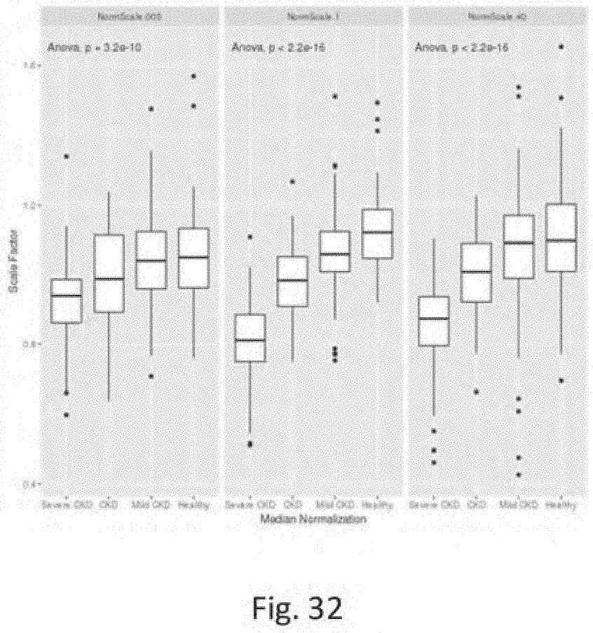

Decreases in GFR result in increases to signals across most analytes, thus, standard median normalization becomes problematic. As the adaptive variable is now continuous the analysis was done by segmenting the data by GFR rates (>90 healthy, 60-90 mild disease, 40-60 disease, 0-40 severe disease) and passing these groups within the adaptive normalization procedure. With standard median normalization we observe significant differences of median normalization scale factors by disease (GFR) state across all dilutions, indicating a strong inverse correlation between GFR and protein levels in plasma. FIG. 32 illustrates standard median normalization scale factors by dilution and disease state partitioned by GFR value. Although this effect exists in all three dilutions, it is weakest in the 0.005% mix, suggesting some of the observed bias is due to NSB as in the example above.

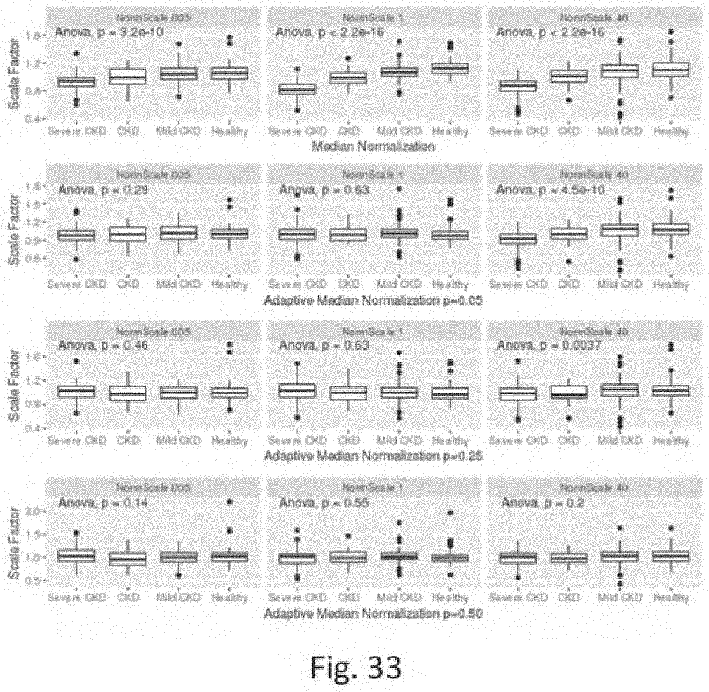

Using adaptive normalization with the disease related directed groups and a p=0.05 cutoff, 738 (of 1211), or 61% of analyte measurements were excluded from median normalization. The number of analytes removed from normalization increases to 1081 (89%) and 1147 (95%) at p=0.25 and p=0.5, respectively. As in the two other studies, adaptive normalization removed correlations of the scale factors with disease severity in the 0.005% and 1% dilutions using a conservative cutoff value of p=0.05, although residual, yet significantly reduced, correlation remains within the 40% dilution. At p=0.5 we have removed all the GFR bias but at the expense of having excluded nearly 95% of all analytes from median normalization. FIG. 33 illustrates median normalization scale factors by dilution and disease state by standard median normalization (top) and adaptive normalization by cutoff.

When the assumptions for standard median normalization are invalid, artifacts will be introduced into the data using standard median normalization. In this extreme case, where a large portion of analyte measurements are correlated with GFR, standard median normalization will attempt to force all measurements to appear to be drawn from the same underlying distribution, thus removing analyte correlations with GFR and decreasing the sensitivity of an analysis. Additional distortions are introduced by moving analyte signals that are unaffected by biology as a consequence of “correcting” the higher signaling analytes in CKD. These distortions are observed as analytes with positive correlation between protein levels and GFR, opposite the true biological signal.

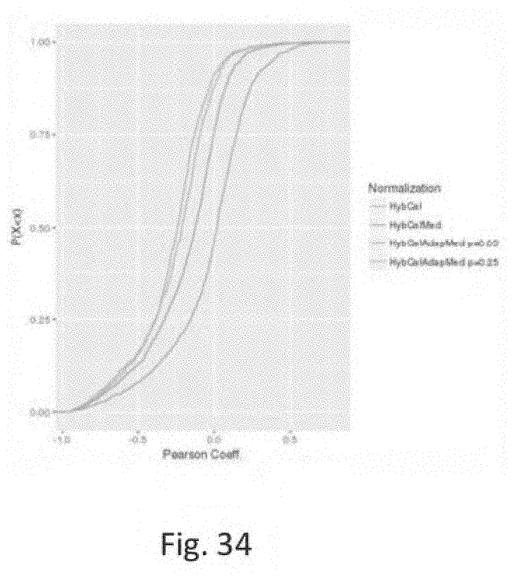

FIG. 34 illustrates this with the CDF of Pearson correlation of all analytes with GFR (log/log) for various normalization procedures. Standard median normalization (HybCalMed) shifts the distribution towards 0—introducing false positive correlations between analyte signals and GFR. Using adaptive normalization reduces this effect as a function of the chosen cutoff value.

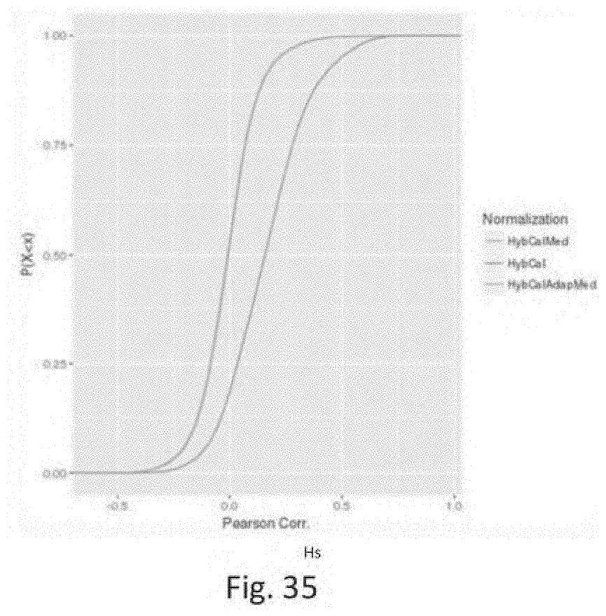

In addition to preserving the true biological correlations between GFR and analyte levels, adaptive normalization also removes the assay induced protein-protein correlations resulting from the correlated noise in the aptamer-based proteomic assay, as shown in FIG. 31. The distribution of inter-protein Pearson correlations for the CKD data set for unnormalized data, standard median normalization and adaptive normalization are presented in FIG. 35.

The unnormalized data show inter-protein correlations centered on ˜0.2 and ranging from ˜−0.3 to +0.75. In the normalized data, these correlations are sensibly centered at 0.0 and range from −0.5 to +0.5. Although many spurious correlations are removed by adaptive normalization, the meaningful biological correlations are preserved since we've already demonstrated that adaptive normalization preserves the physiological correlations with protein levels and GFR.

PBAN Method Analysis

The use of population-based adaptive normalization relies on the meta data associated with a dataset. In practice, it moves normalization from a standard data workup process into an analysis tool when clinical variables, outcomes, or collection protocols affect large numbers of analyte measurements. We've examined studies that have pre-analytical variation as well as an extreme physiological variation and the procedure performs well using bias in the scale factors as a measure of performance.

Aptamer-based proteomic assay data standardization, consisting of hybridization normalization, plate scaling, calibration, and standard median normalization likely suffices for samples collected and run in-house using well-adhered to SomaLogic sample collection and handling protocols. For samples collected remotely, such as the four sites used in the Covance study, this standardization protocol does not hold, as samples can show significant site differences (presumably from comparable sample populations between sites). Each clinical sample set needs to be examined for bias in median normalization scale factors as a quality control step. The metrics explored for such bias should include distinct sites if known as well as any other clinical variate that may result in violations of the basic assumptions for standard median normalization.

The Covance example illustrates the power of the adaptive normalization methodology. In the case of serum samples, little site-dependent bias was observed in the standard median normalization scale factors and the adaptive normalization procedure essentially reproduces the standard median normalization results. But in the case of Covance plasma samples, extreme bias was observed in the standard median normalization scale factors. The adaptive normalization procedure results in normalizing the data without introducing artifacts in the analyte measurements unaffected by the collection differences. The power of the adaptive normalization procedure lies in its ability to normalize data from well collected samples with few biomarkers as well as data from studies with severe collection or biological effects. The methodology easily adapts to include all the analytes that are unaffected by the metrics of interest while excluding only those analytes that are affected. This makes the adaptive normalization technique well suited for application to most clinical studies.

Besides guarding against introducing normalization artifacts into the aptamer-based proteomic assay data, the adaptive normalization method removes spurious correlation due to the correlated noise observed in raw aptamer-based proteomic assay data. This is well illustrated in the CKD dataset where the unnormalized correlations are centered to 0.0 while the important biological correlations with protein levels and GFR are well preserved.

Lastly, adaptive normalization works by removing analytes from the normalization calculation that are not consistent across collection sites or are strongly correlated with disease state, but such differences are preserved and even enhanced after normalization. This procedure does not “correct” collection site bias, or protein levels due to GFR; rather, it ensures that such large differential effects are not removed during normalization since that would introduce artifacts in the data and destroy protein signatures. The opposite is true; most differences are enhanced after adaptive normalization while the undifferentiated measurements are made more consistent.

### CONCLUSIONS

Applicant has developed a robust normalization procedure (population based adaptive normalization, aka PBAN) that reproduces the standard normalization for data sets with consistently collected samples with biological responses involving small numbers of analytes, say <5% of the measurements. For those collections with site dependent bias (pre-analytical variation) or for studies of clinical populations where many analytes are affected, the adaptive normalization procedure guards against introducing artifacts due to unintended sample bias and will not mute biological responses. The analyses presented here support the use of adaptive normalization to guide normalization using key clinical variables or collection sites or both during normalization.

The three normalization techniques described herein have respective advantages. The appropriate technique is contingent on the extent of clinical and reference data available. For example, ANML can be used when the distributions of analyte measurements for a reference population is known. Otherwise, SSAN can be used as an approximation to normalize samples individually. Additionally, population adaptive normalization techniques are useful for normalizing specific cohorts of samples.

The combination of the adaptive and iterative process ensures sample measurements are re-centered around the reference distribution without the potential influence of analyte measurements outside of the reference distribution from biasing scale factors.

Having described and illustrated the principles of our invention with reference to the described embodiment, it will be recognized that the described embodiment can be modified in arrangement and detail without departing from such principles. Elements of the described embodiment shown in software can be implemented in hardware and vice versa.

In view of the many possible embodiments to which the principles of our invention can be applied, we claim as our invention all such embodiments as can come within the scope and spirit of the following claims and equivalents thereto.

1. A method executed by one or more computing devices for adaptive normalization of analyte levels in one or more samples, the method comprising:
receiving, by at least one of the one or more computing devices, one or more analyte levels corresponding to one or more analytes detected in the one or more samples, each analyte level corresponding to a detected quantity of that analyte in the one or more samples; and
normalizing, by at least one of the one or more computing devices, the one or more analyte levels over one or more iterations by, for each iteration, removing any outlier analyte levels in the one or more analyte levels, computing a scale factor based at least in part on at least one remaining analyte level in the one or more analyte levels, and applying the scale factor to the one or more analyte levels;
wherein outlier analyte levels in the one or more analyte levels are determined based at least in part on an outlier analysis between each analyte level and a corresponding reference distribution of that analyte in a reference data set.

2. The method of claim 1, wherein the outlier analysis comprises a distance based outlier analysis.

3. The method of claim 1, wherein the outlier analysis comprises a density based outlier analysis.

4. The method of claim 1, wherein normalizing the one or more analyte levels over one or more iterations comprises performing additional iterations until a change in the scale factor between consecutive iterations is less than or equal to a predetermined change threshold or until a quantity of the one or more iterations exceeds a maximum iteration value.

5. A method executed by one or more computing devices for adaptive normalization of analyte levels in one or more samples, the method comprising:
receiving, by at least one of the one or more computing devices, one or more analyte levels corresponding to one or more analytes detected in the one or more samples, each analyte level corresponding to a detected quantity of that analyte in the one or more samples; and
iteratively applying, by at least one of the one or more computing devices, a scale factor to the one or more analyte levels over one or more iterations until a change in the scale factor between consecutive iterations is less than or equal to a predetermined change threshold or until a quantity of the one or more iterations exceeds a maximum iteration value, each iteration in the one or more iterations comprising:
determining a distance between each analyte level in the one or more analyte levels and a corresponding reference distribution of that analyte in a reference data set;
determining the scale factor based at least in part on analyte levels that are within a predetermined distance of their corresponding reference distributions; and
normalizing the one or more analyte levels by applying the scale factor.

6. The method of claim 5, wherein determining a distance between each analyte level in the one or more analyte levels and a corresponding reference distribution of that analyte in a reference data set comprises:
determining an absolute value of a Mahalanobis distance between each analyte level and the corresponding reference distribution of that analyte in the reference data set.

7. The method of claim 5, wherein determining a distance between each analyte level in the one or more analyte levels and a corresponding reference distribution of that analyte in a reference data set comprises:
determining a quantity of standard deviations between each analyte level and a mean or a median of the corresponding reference distribution of that analyte in the reference data set.

8. The method of claim 5, wherein the predetermined distance comprises a value in a range between 0.5 to 6, inclusive.

9. The method of claim 5, wherein the predetermined distance comprises a value in a range between 1 to 4, inclusive.

10. The method of claim 5, wherein the predetermined distance comprises a value in a range between 1.5 to 3.5, inclusive.

11. The method of claim 5, wherein the predetermined distance comprises a value in a range between 1.5 to 2.5, inclusive.

12. The method of claim 5, wherein the predetermined distance comprises a value in a range between 2.0 to 2.5, inclusive.

13. The method of claim 5, wherein determining the scale factor based at least in part on analyte levels that are within a predetermined distance of their corresponding reference distributions comprises:
determining an analyte scale factor for each analyte level that is within the predetermined distance of the corresponding reference distribution, the analyte scale factor being determined based at least in part on the analyte level and a mean or median value of the corresponding reference distribution;
determining the scale factor by computing either an average or a median of analyte scale factors corresponding to analyte levels that are within the predetermined distance of their corresponding reference distributions.

14. The method of claim 5, wherein determining the scale factor based at least in part on analyte levels that are within a predetermined distance of their corresponding reference distributions comprises:
determining a value of the scale factor that maximizes a probability that analyte levels that are within the predetermined distance of their corresponding reference distributions are part of their corresponding reference distributions.

15. The method of claim 14, wherein the probability that each analyte level is part of the corresponding reference distribution is determined based at least in part on the scale factor, the analyte level, a standard deviation of the corresponding reference distribution, and a median of the corresponding reference distribution.

16. The method of claim 5, wherein the change in the scale factor between subsequent iterations is measured as a percentage change and wherein the predetermined change threshold comprises a value between 0 and 40 percent, inclusive.

17. The method of claim 5, wherein the predetermined change threshold comprises a value between 0 and 20 percent, inclusive.

18. The method of claim 5, wherein the predetermined change threshold comprises a value between 0 and 10 percent, inclusive.

19. The method of claim 5, wherein the predetermined change threshold comprises a value between 0 and 5 percent, inclusive.

20. The method of claim 5, wherein the predetermined change threshold comprises a value between 0 and 2 percent, inclusive.

21. The method of claim 5, wherein the predetermined change threshold comprises a value between 0 and 1 percent, inclusive.

22. The method of claim 5, wherein the predetermined change threshold comprises 0 percent.

23. The method of claim 5, wherein the maximum iteration value comprises one of: 10 iterations, 20 iterations, 30 iterations, 40 iterations, 50 iterations, 100 iterations, or 200 iterations.

24. The method of claim 1, wherein the scale factor is computed by normalizing the at least one remaining analyte level to median or mean values of their corresponding reference distributions.

25. The method of claim 1, wherein the scale factor is computed by maximizing a probability that the remaining analyte levels are part of their corresponding reference distributions.

26. The method of claim 1, wherein the one or more samples comprise a biological sample.

27. The method of claim 26, wherein the biological sample comprises one or more of: a blood sample, a plasma sample, a serum sample, a cerebral spinal fluid sample, a cell lysates sample, or a urine sample.

28. The method of claim 1, wherein the one or more analyte levels corresponding to the one or more analytes detected in the one or more samples comprise a plurality of analyte levels corresponding to a plurality of analytes detected in the one or more samples.

29. The method of claim 1, wherein the one or more analytes comprise one or more of: a protein analyte, a peptide analyte, a sugar analyte, or a lipid analyte

30. The method of claim 1, wherein each analyte level is determined based on applying a binding partner of the analyte to the one or more samples, wherein the binding of the binding partner to the analyte results in a measurable signal, and wherein the measurable signal yields the analyte level.

31. The method of claim 30, wherein the binding partner is an antibody or an aptamer.

32. The method claim 1, wherein each analyte level is determined based on mass spectrometry of the one or more samples.

33. The method of claim 1, wherein the one or more samples comprise a plurality of samples, wherein the one or more analyte levels corresponding to the one or more analytes comprise a plurality of analyte levels corresponding to each analyte, and wherein determining a distance between each analyte level in the one or more analyte levels and a corresponding reference distribution of that analyte in a reference data set comprises:
determining a Student's T-test, Kolmogorov-Smirnov test, or a Cohen's D statistic between the plurality of analyte levels corresponding to each analyte and the corresponding reference distribution of each analyte in the reference data set.

34. At least one non-transitory computer-readable medium for adaptive normalization of analyte levels in one or more samples and storing computer-readable instructions that, when executed by one or more computing devices, cause at least one of the one or more computing devices to:
receive one or more analyte levels corresponding to one or more analytes detected in the one or more samples, each analyte level corresponding to a detected quantity of that analyte in the one or more samples; and
normalize the one or more analyte levels over one or more iterations by, for each iteration, removing any outlier analyte levels in the one or more analyte levels, computing a scale factor based at least in part on at least one remaining analyte level in the one or more analyte levels, and applying the scale factor to the one or more analyte levels;
wherein outlier analyte levels in the one or more analyte levels are determined based at least in part on an outlier analysis between each analyte level and a corresponding reference distribution of that analyte in a reference data set.

35. An apparatus for adaptive normalization of analyte levels in one or more samples, the apparatus comprising:
one or more processors; and
one or more memories operatively coupled to at least one of the one or more processors and having instructions stored thereon that, when executed by at least one of the one or more processors, cause at least one of the one or more processors to:
receive one or more analyte levels corresponding to one or more analytes detected in the one or more samples, each analyte level corresponding to a detected quantity of that analyte in the one or more samples; and
normalize the one or more analyte levels over one or more iterations by, for each iteration, removing any outlier analyte levels in the one or more analyte levels, computing a scale factor based at least in part on at least one remaining analyte level in the one or more analyte levels, and applying the scale factor to the one or more analyte levels;
wherein outlier analyte levels in the one or more analyte levels are determined based at least in part on an outlier analysis between each analyte level and a corresponding reference distribution of that analyte in a reference data set.
